(19) United States
(12) Patent Application Publication
(10) Pub. No.: US 2005/0155102 A1
(43) Pub. Date: Jul. 14, 2005

(54) Methods and compositions for altering the functional properties of seed storage proteins in soybean

(75) Inventors: Darren B. Gruis, Des Moines, IA (US); Rudolf Jung, Des Moines, IA (US)

Correspondence Address: ALSTON & BIRD LLP;PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignee: Pioneer Hi-Bred International, Inc., Johnston, IA (US)

(21) Appl. No.: 11/011,522

(22) Filed: Dec. 14, 2004

Related U.S. Application Data
(60) Provisional application No. 60/529,666, filed on Dec. 15, 2003.

(51) Int. Cl. A01H 1/00; C12N 15/82; A01H 5/00

(52) U.S. Cl. 800/278; 800/312

(57) ABSTRACT

The present invention provides methods and compositions useful for altering the functional properties of soybean seed storage proteins. It is the novel finding of the present invention that the functional properties of seed storage proteins can be altered by reducing the expression of one or more vacuolar processing enzymes in plant seed. Accordingly, in one embodiment, the invention provides a method for altering the functional properties of one or more soybean seed storage proteins. The method comprises transforming a soybean plant cell with at least one expression cassette capable of expressing a polynucleotide that reduces the activity of a vacuolar processing enzyme in the seed of said soybean plant, regenerating a transformed plant from the transformed plant cell, and collecting seed from the regenerated transformed plant. Plants that are genetically modified or mutagenized to alter the functional properties of one or more seed storage proteins, and the transgenic seed of such plants are also provided.

### CROSS-REFERENCE TO RELATED APPLICATIONS

This application claims the benefit of U.S. Provisional Application No. 60/529,666, filed Dec. 15, 2003, which is hereby incorporated in its entirety by reference herein.

### FIELD OF THE INVENTION

The present invention relates to genetic modification of soybean, more particularly to the alteration of the functional properties seed storage proteins in soybean.

### BACKGROUND OF THE INVENTION

Many plant storage tissues (seeds, leaves, roots, and tubers), accumulate sizable reserves of proteins during development. For example, cultivated soybean seeds contain an average of about 40% protein, and in some varieties protein levels reach as much as 55% of the dry weight. The abundance of proteins in legume seeds has made them the primary dietary protein source and has stimulated an interest in developing approaches to genetically engineer seeds to improve their nutritional quality.

Plant storage proteins, especially those processed through the secretory pathway, generally undergo multiple post-translational processing steps including folding, assembly, intracellular sorting, and proteolytic processing, prior to final deposition (Müntz et al., (1993) Proc. Phytochem. Soc. Eur. 35: 128-146; Müntz (1998) Plant Mol. Biol. 38: 77-99; Herman and Larkins (1999) Plant Cell 11: 601-613). Accumulation and deposition of the proteins is accomplished by compartmentalization in specialized vacuoles termed protein storage vacuoles and or protein bodies (Hara-Nishimura et al. (1995) J. Plant Physiol. 145: 632-640; Müntz (1998) Plant Molec. Biol. 38: 77-99; Herman and Larkins (1999) Plant Cell 11: 601-613).

The proteolytic processing steps of protein deposition in vacuoles include specific polypeptide cleavage steps accomplished by proteases localized to the storage vacuole (Bassham et al. (2000) Curr. Opin. Cell Biol. 12: 491-495). Storage proteins that accumulate in vacuoles have therefore co-evolved with the environment of the storage vacuole, such that only a select few protease sites exist or are accessible to these proteases (Hara-Nishimura et al. (1987) Plant Physiol. 85: 440-445; D'Hondt et al., (1993) J. Biol. Chem. 268: 10884-10891; Hara-Nishimura et al. (1993) Plant Cell 5: 1651-1659; Hara-Nishimura et al. (1995) J. Plant Physiol. 145: 632-640).

Glycinin is a major soybean seed storage protein that is used extensively in soy food products. However, this protein's functional properties limit its use in some product applications. For example, glycinin is insoluble at low pH, and so it is not well suited for use in acidic food products. See, for example, Lakemond et al. (2000) J. Agric. Food Chem. 48: 1985-90 and Mohamed et al. (2002) J. Agric. Food Chem. 50: 7380-85.

Accordingly, methods are needed to alter the functional properties of seed storage proteins in soybeans.

### SUMMARY OF THE INVENTION

The present invention is directed to altering the functional properties of soybean seed storage proteins. It is the novel finding of the present invention that the functional properties of seed storage proteins can be altered by reducing the expression of one or more vacuolar processing enzymes (VPEs) in plant seed. Accordingly, in one embodiment, the invention provides a plant that is genetically modified to alter one or more functional properties of one or more seed storage proteins. The invention also provides methods for altering the functional properties of one or more soybean seed storage proteins. In some embodiments, the method comprises transforming a soybean plant cell with at least one expression cassette capable of expressing a polynucleotide that reduces or eliminates the activity of a vacuolar processing enzyme in the seed of said soybean plant, regenerating a transformed plant from the transformed plant cell, and collecting seed from the regenerated transformed plant. In other embodiments, the method comprises transforming a soybean plant cell with at least one expression cassette comprising a polynucleotide encoding a polypeptide that reduces or eliminates the activity of at least one vacuolar processing enzyme in seed in the seed of said soybean plant, regenerating a transformed plant from the transformed plant cell, and collecting seed from the regenerated transformed plant.

According to the invention, the activity of at least one, at least two, at least three, at least four, at least five, or at least six vacuolar processing enzymes may be reduced or eliminated in soybean seed. Thus, the soybean plants may be transformed with two or more polynucleotides, which inhibit the expression of a soybean vacuolar processing enzyme. In some embodiments, the polynucleotide is designed to reduce or eliminate the activity of only one vacuolar processing enzyme, while in other embodiments the polynucleotide is designed to reduce or eliminate the expression of two or more different soybean vacuolar processing enzymes, three or more different soybean vacuolar processing enzymes, or more than three different soybean vacuolar processing enzymes. When two or more polynucleotides are transformed into the same plant cell, they may be expressed from the same expression cassette. Alternatively, the polynucleotides may be comprised in separate expression cassettes.

In some embodiments, at least one of the soybean vacuolar processing enzymes whose activity is reduced or eliminated is selected from the group consisting of soybean Vpe1a, Vpe1b, Vpe2a, Vpe2b, Vpe3a, and Vpe3b. In further embodiments, at least one vacuolar processing enzyme whose expression is inhibited is selected from the group consisting of SEQ ID NO:2, SEQ ID NO:4, SEQ ID NO:6, SEQ ID NO:8, SEQ ID NO:10, SEQ ID NO:12, and SEQ ID NO:14.

In certain embodiments, at least one functional property that is altered in the seed storage protein is the solubility of the seed storage protein. In particular embodiments, the solubility of a seed storage protein is increased at low pH. For example, the invention provides embodiments in which the solubility of the seed storage protein is increased between pH 4.0 and pH 6.0.

In some embodiments, the soybean seed storage protein whose functional properties are altered is selected from glycinin, soybean 2S albumin, and β-conglycinin.

The expression cassettes used in the method of invention may be any expression cassette capable of reducing or eliminating the expression of at least one soybean vacuolar processing enzyme.

The invention also provides soybean plants that are genetically modified to alter the functional properties of one or more seed storage proteins. In some embodiments, the soybean plant is genetically modified to reduce or eliminate the expression of one or more vacuolar processing enzymes in seed. In particular embodiments, the soybean plant is stably transformed with an expression cassette capable of expressing at least one polynucleotide that inhibits the expression of a vacuolar processing enzyme in seed. In other embodiments, the soybean plant is stably transformed with at least one polynucleotide comprising a polynucleotide encoding a polypeptide that inhibits the activity of a vacuolar processing enzyme.

The soybean plant of the invention may be genetically modified to reduce or eliminate the activity of at least one, at least two, at least three, at least four, at least five, at least six, or at least seven or more soybean vacuolar processing enzymes. Transgenic seed of the genetically modified plant is also encompassed.

The invention also encompasses soybean vacuolar processing enzymes and polynucleotides encoding these vacuolar processing enzymes. Polypeptides of the invention include those having the sequence shown in SEQ ID NOS:2, 4, 6, 8, and 10, as well as variants and fragments thereof. Polynucleotides of the invention include those having the sequence shown in SEQ ID NOS:1, 3, 5, 7, and 9, as well as variants and fragments thereof.

### BRIEF DESCRIPTION OF THE DRAWINGS

FIG. 1 shows the solubility properties of legumin-type globulin protein isolated from mature wild-type and vpe-quad Arabidopsis seeds. Legumin-type globulin was isolated from sucrose density gradients. Solubility of protein obtained from these fractions was determined under low ionic strength conditions at various pH. Following incubation of the protein sample at a given pH, the amount of protein remaining in solution was quantified and graphed as a percent of the total protein added to the reaction. The error bars show standard deviations (3 replications) at each data point.

FIG. 2 shows the solubility profiles for normally processed glycinin (Native Gly 11S) isolated from soybean seed and of the unprocessed proglycinin protein, obtained by expression of an appropriate expression construct in bacterial cells. The unprocessed glycinin pro-protein has much greater solubility than the native (processed) glycinin between pH 4.5 and pH 5.5.

### DETAILED DESCRIPTION OF THE INVENTION

The present invention provides methods and compositions useful for altering the functional properties of soybean seed storage proteins. It is the novel finding of the present invention that the functional properties of seed storage proteins can be altered by reducing the expression and/or activity of one or more vacuolar processing enzymes in plant seed. Accordingly, the invention provides methods for altering the properties of soybean seed storage proteins by reducing or eliminating the activity of one or more endogenous vacuolar processing enzymes in soybean seed, soybean plants with altered functional properties for one or more seed storage proteins, and compositions comprising soybean seed storage proteins produced by the methods of the invention.

In some embodiments, the method comprises the steps of transforming a soybean plant cell with at least one expression cassette capable of expressing a polynucleotide that reduces of eliminates the activity of at least one soybean vacuolar processing enzyme, regenerating a transformed plant from the transformed plant cell, and collecting seed from the regenerated transformed plant.

In additional embodiments, the method comprises the steps of transforming a soybean plant cell with at least one expression cassette comprising a polynucleotide encoding a polypeptide that reduces of eliminates the activity of at least one soybean vacuolar processing enzyme, regenerating a transformed plant from the transformed plant cell, and collecting seed from the regenerated transformed plant. The seed harvested from the transformed plant contains seed storage proteins having altered functional properties.

The invention also provides soybean seed storage proteins having altered functional properties, and compositions comprising these storage proteins.

Also provided are plants that are genetically modified or mutagenized to reduce or eliminate the activity of one or more soybean vacuolar processing enzymes, and transformed seed of these plants.

The methods and compositions of the invention are described in more detail below.

### Soybean Seed Storage Proteins

The invention relates to methods of altering the functional properties of one or more seed storage proteins in soybean, and to soybean plants that are genetically modified or mutagenized to alter the functional properties of one or more seed storage proteins. The functional properties of any soybean seed storage protein may be altered according to the invention. Soybean has three major seed storage proteins; two globulins, glycinin (also known as the 11S globulins) and β-conglycinin (also known as the 7S globulins), and one albumin, 2S albumin. Together, these proteins comprise 70% to 80% of the soybean seed's total protein, or 25 to 35% of the seed's dry weight. Glycinin is a large protein with a molecular weight of about 360 kDa. It is a hexamer composed of the various combinations of five different types of subunits, which are identified as G1, G2, G3, G4 and G5. Each subunit is composed of one acidic region and one basic region held together by a disulfide bond. The glycinin subunits are primarily encoded by genes designated Gy1, Gy2, Gy3, Gy4 and Gy5, corresponding to subunits G1, G2, G3, G4 and G5, respectively (Nielsen, N. C. et al. (1989) Plant Cell 1: 313-328). At least one other gene, Gy7, also appears to encode a glycinin subunit (Beilinson et al. (2002) Theor. Appl. Genet. 104: 1132-40).

β.-conglycinin is a heterogeneously glycosylated protein with a molecular weight ranging from 150 and 240 kDa. It is composed of varying combinations of three highly negatively charged subunits identified as α, α, and β. The three classes of β-conglycinin subunits are encoded by a total of 15 subunit genes clustered in several regions within the genome soybean (Harada, J. J. et al. (1989) Plant Cell 1: 415-425).

The sulfur-rich 2S albumin comprises between 5-10% of the soybean seed's total protein. See, NCBI Accession No. AF005030, U.S. Pat. No. 5,850,016, and Alfredo et al. (1997) Plant Physiol. 114: 1567, each of which is herein incorporated by reference.

Over the past 20 years, significant effort has been aimed at understanding the functional properties of soybean seed storage proteins. See, for example, Kinsella et al. (1985) New Protein Foods 5: 107-179; Morr (1987) JAOCS 67: 265-271; and Peng et al. (1984) Cereal Chem. 61: 480-489. Examples of functional properties of interest include solubility, water adsorption, binding, and retention, gelation (including gel firmness), cohesion-adhesion, elasticity, emulsification, fat-adsorption, flavor binding, foaming, and color control. See, for example, Kinsella (1979) J. Amer. Oil Chemists Soc. 56: 242-58, herein incorporated by reference. The present invention relates the alteration of the functional properties of soybean seed storage proteins, such as the solubility, water retention properties, gelation properties, or emulsification properties of soybean seed storage proteins. These functional properties are related, and thus an alteration in one functional property (such as solubility) can lead to an alteration in other functional properties. Thus, in some embodiments, one functional property is altered, while in other embodiments, multiple functional properties such as two or more functional properties, three or more functional properties, or four or more functional properties are altered.

In some embodiments, the gelation properties of one or more soybean storage proteins are altered. By “gelation properties” it is intended the ability of a protein to form a three-dimensional matrix of intertwined, partially associated polypeptides in which water can be held. See, for example, Kinsella (1979) J. Amer. Oil Chemists Soc. 56: 242-58, herein incorporated by reference.

In some embodiments, the emulsification properties of one or more soybean storage proteins are altered. By “emulsification properties” it is intended the ability of a protein to aid in the uniform formation and stabilization of fat emulsions. See, for example, Kinsella (1979) J. Amer. Oil Chemists Soc. 56: 242-58, herein incorporated by reference.

In some embodiments, the water retention properties of one or more soybean storage proteins are altered. Water retention of soybean protein isolates is dependent in part on the proteolyzed state of the proteins in the isolate (Mietsch et al. (1989) Nahrung 33: 9-15).

In some embodiments, the solubility of one or more soybean seed storage proteins is altered. By “solubility” it is intended dispersibility in fluid. Solubility may be measured using the nitrogen solubility index (NSI) or the protein dispersibility index. See, Johnson (1970) Food. Prod. Dev. 3: 78, and Johnson (1970) JAOCS 47: 402; both of which are herein incorporated by reference in their entireties. The solubility of a protein solution can be measured by centrifuging the solution at 17,000×g for 10 minutes, and then assaying the supernatant to determine protein content.

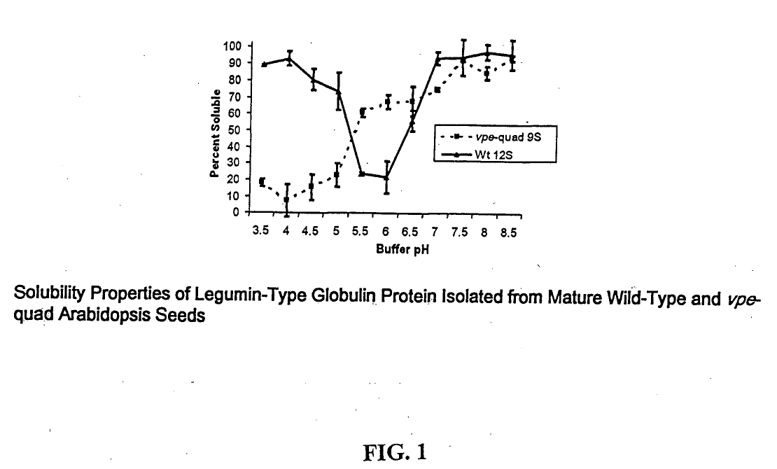

It is the novel finding of the present invention that eliminating the expression of vacuolar processing enzymes in seed results in a marked alteration in the solubility of seed storage proteins. The legumin-like seed storage proteins of Arabidopsis are relatively insoluble at low pH, having less than 20% solubility in solutions having a pH of less than 5, and only about 25% solubility at pH 5.5. However, in an Arabidopsis plant null for α, β, γ, and δ vacuolar processing enzymes, the legumin-type globulin proteins show greatly enhanced solubility between pH 3.5 and pH 5.0. See FIG. 1 and the Experimental section.

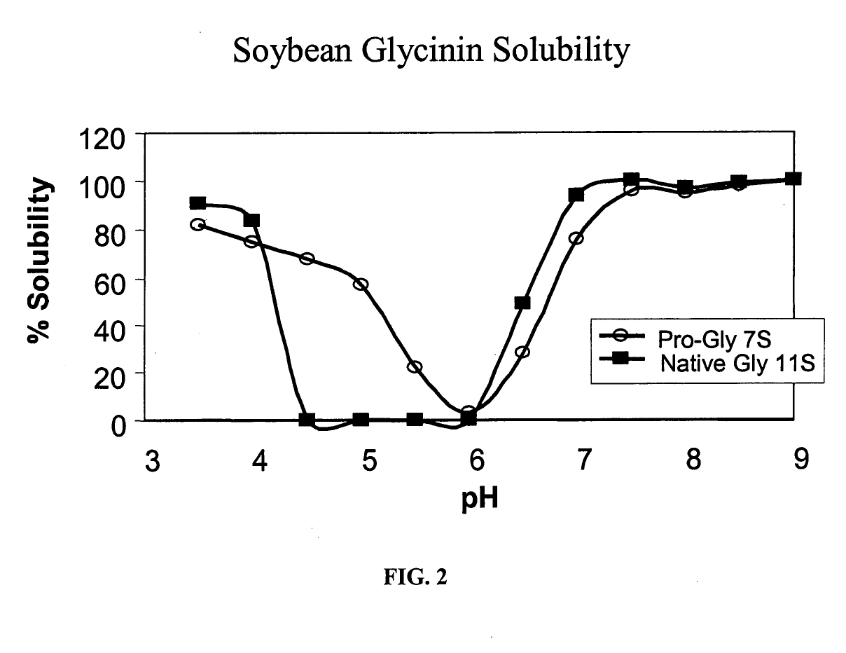

The present invention also shows that soybean glycinin proteins that are not cleaved by vacuolar processing enzymes have increased solubility at low pH in comparison with glycinin that is cleaved by vacuolar processing enzymes. See, FIG. 2. Accordingly, reducing the expression of soybean vacuolar processing enzymes increases the solubility of glycinin in soybean seed.

Thus, in some embodiments, the present invention provides methods of producing a soybean seed storage protein having increased solubility, and soybean plants that have been genetically modified to increase the solubility of a seed storage protein. A seed storage protein in a plant that has been genetically modified to inhibit the expression of one or more vacuolar processing enzymes has increased solubility according to the invention if the solubility of the protein is at least 2 times greater than the solubility of the same protein in a plant that has not been genetically modified to inhibit the expression of a vacuolar processing enzyme. In some embodiments, the solubility of the soybean seed storage protein in a plant that has been genetically modified to inhibit the expression of one or more vacuolar processing enzymes is at least 5 times greater than, at least 10 times greater than, at least 20 times greater than, at least 50 times greater than, at least 100 times greater than, or more than 100 times greater than the solubility of the same protein in a plant that has not been genetically modified to inhibit the expression of a vacuolar processing enzyme.

In some embodiments of the invention, the solubility of a seed storage protein is increased at low pH. For example, the invention provides embodiments in which the solubility of the seed storage protein is increased in the pH range between pH 3.5 and pH 6.5. In particular embodiments, the solubility of the seed storage protein is increased between pH 4.0 and 6.0, such as between pH 4.5 and 5.5. Soybean seed storage proteins having increased solubility according to the invention will be at least 10% soluble, at least 20% soluble, at least 30% soluble, at least 40% soluble, at least 50% soluble, at least 60% soluble, at least 70% soluble, at least 80% soluble, or more than 80% soluble in solutions having a pH ranging between 4.5 and 5.5. In some embodiments, one or more of the seed storage proteins is glycinin. In another embodiment one or more of the seed storage proteins is 2S albumin.

The invention also encompasses soybean seed storage proteins having altered functional properties, and compositions comprising these seed storage proteins. Soy protein products are generally categorized into three major groups: soy flours and grits containing about 45 to 54% soy protein on a moisture free basis; soy protein concentrates containing 65 to 90% protein on a moisture free basis; and soy protein isolates having a minimum of 90% protein on a moisture free basis. Soy protein isolates are preferred in many applications because of their higher protein content, easier digestibility, and improved flavor as compared with soy flours, grits and concentrates. In one embodiment, the invention pertains to the production of soy protein isolates, which are the most highly refined soy protein products commercially available.

### Soybean Vacuolar Processing Enzymes

According to the invention, the proteolytic activity of at least one, at least two, at least three, at least four, at least five, at least six, or at least seven, or more than seven vacuolar processing enzymes may be reduced or eliminated in soybean seed. In plants, vacuolar processing enzymes (VPE's) comprise a small gene family of plant asparaginyl endopeptidases implicated in the control of several important cellular processes including storage protein proteolysis involved in protein turnover and mobilization of amino acid reserves in vegetative tissue during plant senescence process. See, for example, Hara-Nishimura et al. (1987) Plant Physiol 85: 440-445; D'Hondt et al. (1993) J. Biol. Chem. 268: 20884-20891; Hara-Nishimura et al. (1993) Plant Cell 5: 1651-1659; Hara-Nishimura et al. (1995) J. Plant Physiol. 145: 632-640; and Kinoshita et al. (1995) Plant Cell Physiol. 36: 1555-1562; D'Hondt et al. (1997) Plant Molec. Biol. 33: 187-192; Barrett et al., ed. (1998) Handbook of Proteolytic Enzymes, Academic Press, Sand Diego, pp 746-749, each of which is incorporated by reference.

Vacuolar processing enzymes are a member of peptidase family C13 (see Pfam Accession number PF01650), and catalyze the hydrolysis of proteins at -Asn-|-Xaa peptide bonds. These cysteine proteases are members of enzyme class 3.4.22.34. Alternate names for this family include legumain, asparaginyl endopeptidase, phaseolin endopeptidase, and bean endopeptidase. This family of peptidases is described, for example, in Hara-Nishimura, Asparinyl endopeptidase in Handbook of Proteolytic Enzymes, Barrett et al., eds., pp. 746-749 (1998) Academic Press, London; Dalton and Brindley, Schistosome Legumain in Handbook of Proteolytic Enzymes, Barrett et al., eds., pp. 749-754 (1998) Academic Press, London; Chen et al. (1998) FEBS Letters 441: 361-65, and Muntz and Shutov (2002) Trends in Plant Science 7: 340-44; each of which is herein incorporated by reference.

By a “soybean vacuolar processing enzyme” as used herein, it is intended a soybean cysteine protease that is a member of the peptidase C13 family (Pfam Accession number PF01650) and has the proteolytic activity of enzyme class 3.4.22.34, i.e. the ability catalyze the hydrolysis of proteins at -Asn-|-Xaa-peptide bonds. See Chen et al. (1998) FEBS Letters 441: 361-365 for a description of active site residues involved in vacuolar processing enzyme activity. See Jung et al. (1998) The Plant Cell 10: 343-57, herein incorporated by reference, for a description of the substrate specificity of soybean vacuolar processing enzymes in soybean and for assays for determining vacuolar processing enzyme activity.

The present invention provides amino acid sequences for soybean Vpe1a (SEQ ID NO:2), Vpe1b (SEQ ID NO:4), Vpe2a (SEQ ID NO:6), Vpe2b (SEQ ID NO:8), and Vpe3a (SEQ ID NO:110). Nucleotide sequences encoding these soybean VPEs are set forth in SEQ ID NO:1 (Vpe1 a), SEQ ID NO:3 (Vpe1b), SEQ ID NO:5 (Vpe2a), SEQ ID NO:7 (Vpe2b), and SEQ ID NO:9 (Vpe3a).

Soybean vacuolar processing enzymes (VPE's) have been also described in the art. See, for example, the soybean VPE described by Shimada et al. (11994) Plant Cell Physiol. 35: 713-718. The coding sequence for this soybean VPE is set forth as SEQ ID NO:11, and the encoded protein is set forth in SEQ ID NO:12. See also NCBI Accession number AF169019. The coding sequence for this soybean VPE is set forth as SEQ ID NO:13, and the encoded protein is set forth in SEQ ID NO:14.

The soybean VPE's can be grouped phylogentically into gene sub families, as has been described for members of the VPE gene family of other plants (Muntz and Shutov (2002) Trends in Plant Science 7: 340-44). Soybean Vpe1a and Vpe1b are seed-type VPE's and are closely related to β-VPE from Arabidopsis, while Vpe2a, Vpe2b, Vpe3a, and Vpe3b are vegetative-type VPE's and closely related to α- and γ-VPE from Arabidopsis.

Thus, in some embodiments of the invention, at least one of the vacuolar processing enzymes whose activity is reduced is selected from the group consisting of Vpe1a, Vpe1b, Vpe2a, Vpe2b, Vpe3a, and Vpe3b. In further embodiments, at least one vacuolar processing enzyme whose expression is inhibited is selected from the group consisting of SEQ ID NO:2, SEQ ID NO:4, SEQ ID NO:6, SEQ ID NO:8, SEQ ID NO:10, SEQ ID NO:12, and SEQ ID NO:14.

The invention encompasses the inhibition of the expression of soybean homo logs of the proteins set forth in SEQ ID NOS:2, 4, 6, 8, 10, 12, and 14. Such soybean homologs typically have substantial sequence similarity with at least one amino acid sequence selected from SEQ ID NOS:2, 4, 6, 8, 10, 12, and 14, and the nucleotide sequences encoding them typically have substantially similarity to at least one nucleotide sequence selected from SEQ ID NOS:1, 3, 5, 7, 9, 11, and 13. The homologs also have the protease activity of a protein set forth in SEQ ID NO:2, 4, 6, 8, 10, 12, or 14, i.e., the homologs catalyze the hydrolysis of proteins at -Asn-|-Xaa-peptide bonds. Thus in some embodiments, the invention comprises inhibiting the expression of a soybean vacuolar protease encoded by a sequence having at least 70% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, or more than 99% sequence identity with at least one nucleotide sequence selected from SEQ ID NOS:1, 3, 5, 7, 9, 11, and 13. Methods of calculating the level of sequence identity between two sequences are provided elsewhere herein.

The proteolytic activity of a soybean vacuolar processing enzyme may determined by any method known in the art. Methods for determining the proteolytic activity of a vacuolar processing enzyme are described, for example, in Jung et al. (1998) The Plant Cell 10: 343-57, Hara-Nishimura, Asparinyl endopeptidase in Handbook of Proteolytic Enzymes, Barrett et al., eds., pp. 746-749 (1998) Academic Press, London; and Dalton and Brindley, Schistosome Legumain in Handbook of Proteolytic Enzymes, Barrett et al., eds., pp. 749-754 (1998) Academic Press, London; Chen et al. (1998) FEBS Letters 441: 361-65; each of which is herein incorporated by reference.

The invention also encompasses soybean vacuolar processing enzymes and nucleotide sequences encoding soybean vacuolar processing enzymes. In particular, the present invention provides for isolated polynucleotide comprising nucleotide sequences encoding the amino acid sequences shown in SEQ ID NOS:2, 4, 6, 8, and 10. Further provided are polypeptides having an amino acid sequence encoded by a polynucleotide described herein, for example those set forth in SEQ ID NOS:1, 3, 5, 7, and 9.

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of a polynucleotide may encode protein fragments that retain the protease activity of the native vacuolar processing enzyme. Alternatively, fragments of a polynucleotide that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the proteins of the invention.

A fragment of a vacuolar processing enzyme polynucleotide that encodes a biologically active portion of a vacuolar processing enzyme of the invention will encode at least 15, 25, 30, 50, 100, 150, 200, 250, 300, 350, 400, or 450 contiguous amino acids, or up to the total number of amino acids present in a full-length vacuolar processing enzyme of the invention (for example, 495 amino acids for SEQ ID NO:2, SEQ ID NO:4, 484 amino acids for SEQ ID NO:6, 483 amino acids for SEQ ID NO:8, or 482 amino acids for SEQ ID NO:10, respectively). Fragments of a soybean vacuolar processing enzyme polynucleotide that are useful as hybridization probes or PCR primers generally need not encode a biologically active portion of a vacuolar processing enzyme.

Thus, a fragment of a vacuolar processing enzyme polynucleotide may encode a biologically active portion of a vacuolar processing enzyme, or it may be a fragment that can be used as a hybridization probe or PCR primer using methods disclosed below. A biologically active portion of a vacuolar processing enzyme can be prepared by isolating a portion of one of the vacuolar processing enzyme polynucleotide of the invention, expressing the encoded portion of the vacuolar processing enzyme (e.g., by recombinant expression in vitro), and assessing the protease activity of the encoded portion of the vacuolar processing enzyme. Polynucleotides that are fragments of a vacuolar processing enzyme nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, or 1,700 contiguous nucleotides, or up to the number of nucleotides present in a full-length vacuolar processing enzyme polynucleotide disclosed herein (for example, 1769 nucleotides for SEQ ID NO:1, 1806 nucleotides for SEQ ID NO:3, 1936 nucleotides for SEQ ID NO:5, 1942 nucleotides for SEQ ID NO:7, or 1948 nucleotides for SEQ ID NO:9.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the vacuolar processing enzymes of the invention, as well as naturally occurring allelic variants. Naturally occurring allelic variants can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode a vacuolar processing enzyme of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NOS:2, 4, 6, 8, or 10 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, that is, protease activity as described herein. Such variants may result from, for example, genetic polymorphism or from human manipulation. Biologically active variants of a native vacuolar processing enzyme of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants and fragments of the vacuolar processing enzymes can be prepared by mutations in the DNA. Methods for mutagenesis and polynucleotide alterations are well known in the art. See, for example, Kunkel (1985) Proc. Natl. Acad. Sci. USA 82: 488-492; Kunkel et al. (1987) Methods in Enzymol. 154: 367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired protease activity. Obviously, the mutations that will be made in the DNA encoding the variant must not place the sequence out of reading frame and optimally will not create complementary regions that could produce secondary miRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays for vacuolar processing enzyme activity as described herein.

Variant polynucleotides and proteins also encompass sequences derived from a mutagenic and recombinogenic procedure such as DNA shuffling. With such a procedure, one or more different vacuolar processing enzyme coding sequences can be manipulated to create a new vacuolar processing enzyme possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91: 10747-10751; Stemmer (1994) Nature 370: 389-391; Crameri et al. (1997) Nature Biotech. 15: 436-438; Moore et al. (1997) J. Mol. Biol. 272: 336-347; Zhang et al. (1997) Proc. Natl. Acad. Sci. USA 94: 4504-4509; Crameri et al. (1998) Nature 391: 288-291; and U.S. Pat. Nos. 5,605,793 and 5,837,458.

### Methods of Reducing the Proteolytic Activity of Vacuolar Processing Enzymes

The present invention encompasses methods of producing one or more seed storage proteins having altered functional properties by reducing or eliminating the proteolytic activity of one or more vacuolar processing enzymes. The invention also encompasses soybean plants that have been genetically modified or mutagenized to reduce or eliminate the activity of one or more vacuolar processing enzymes.

In some embodiments, the activity of the vacuolar processing enzyme is reduced or eliminated by transforming a soybean plant cell with an expression cassette that expresses a polynucleotide that inhibits the expression of the vacuolar processing enzyme. The polynucleotide may inhibit the expression of one or more vacuolar processing enzymes directly, by preventing translation of the vacuolar processing enzyme messenger RNA, or indirectly, by encoding a polypeptide that inhibits the transcription or translation of a soybean gene encoding a vacuolar processing enzyme. Methods for inhibiting or eliminating the expression of a gene in a plant are well known in the art, and any such method may be used in the present invention to inhibit the expression of one or more soybean vacuolar processing enzymes.

The expression of a vacuolar processing enzyme is inhibited according to the present invention if the protein level of the vacuolar processing enzyme is less than 70% of the protein level of the same vacuolar processing enzyme in a plant that that has not been genetically modified or mutagenized to inhibit the expression of that vacuolar processing enzyme. In particular embodiments of the invention, the protein level of the vacuolar processing enzyme in a modified plant according to the invention is less than 60%, less than 50%, less than 40%, less than 30%, less than 20%, less than 10%, or less than 5% than of the protein level of the same vacuolar processing enzyme in a plant that this is not a mutant or that has not been genetically modified to inhibit the expression of that vacuolar processing enzyme. The expression level of the vacuolar processing enzyme may be measured directly, by assaying for the level of vacuolar processing enzyme expressed in the soybean cell or plant, or indirectly, by measuring the proteolytic activity of the vacuolar processing enzyme in the soybean cell or plant. Methods for determining the proteolytic activity of vacuolar processing enzymes are described elsewhere herein.

In other embodiments of the invention, the activity of one or more soybean vacuolar processing enzymes is reduced or eliminated by transforming a soybean plant cell with an expression cassette comprising a polynucleotide encoding a polypeptide that inhibits the activity of one or more soybean vacuolar processing enzymes. The proteolytic activity of a vacuolar processing enzyme is inhibited according to the present invention if the proteolytic activity of the vacuolar processing enzyme is less than 70% of the proteolytic activity of the same vacuolar processing enzyme in a plant that has not been genetically modified to inhibit the proteolytic activity of that vacuolar processing enzyme. In particular embodiments of the invention, the proteolytic activity of the vacuolar processing enzyme in a modified plant according to the invention is less than 60%, less than 50%, less than 40%, less than 30%, less than 20%, less than 10%, or less than 5% than of the proteolytic activity of the same vacuolar processing enzyme in a plant that that has not been genetically modified to inhibit the expression of that vacuolar processing enzyme. The proteolytic activity of a vacuolar processing enzyme is “eliminated” according to the invention when it is not detectable by the assay methods described elsewhere herein. Methods of determining the proteolytic activity of a vacuolar processing enzyme are described elsewhere herein.

In other embodiments, the activity of a vacuolar processing enzyme may be reduced or eliminated by disrupting the gene encoding the vacuolar processing enzyme. The invention encompasses mutagenized soybean plants that carry mutations in VPE genes, where the mutations reduce expression of the VPE genes or inhibit the proteolytic activity of the encoded VPE.

Thus, many methods may be used to reduce or eliminate the activity of a vacuolar processing enzyme. More than one method may be used to reduce the activity of a single soybean vacuolar processing enzyme. In addition, combinations of methods may be employed to reduce or eliminate the activity of two or more different vacuolar processing enzymes, three or more different vacuolar processing enzymes, four or more different vacuolar processing enzymes, five or more different vacuolar processing enzymes, or six or more different vacuolar processing enzymes.

Non-limiting examples of methods of reducing or eliminating the expression of a soybean vacuolar processing enzyme are given below.

I. Polynucleotides that Inhibit the Expression of One or More Vacuolar Processing Enzymes

In some embodiments of the present invention, a soybean plant cell is transformed with an expression cassette that is capable of expressing a polynucleotide that inhibits the expression of one or more vacuolar processing enzymes. The term “expression” as used herein refers to the biosynthesis of a gene product, including the transcription and/or translation of said gene product. For example, for the purposes of the present invention, an expression cassette capable of expressing a polynucleotide that inhibits the expression of at least one soybean vacuolar processing enzyme is an expression cassette capable of producing an RNA molecule that inhibits the transcription and/or translation of at least one soybean vacuolar processing enzyme. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a soybean vacuolar processing enzyme are given below.

A. Sense Suppression/Cosuppression

In some embodiments of the invention, inhibition of the expression of a vacuolar processing enzyme may be obtained by sense suppression or cosuppression. For cosuppression, the expression cassette is designed to express an RNA molecule corresponding to all or part of a messenger RNA encoding a soybean vacuolar processing enzyme in the “sense” orientation. Over expression of the RNA molecule can result in reduced expression of the native gene. Accordingly, multiple plants lines transformed with the cosuppression expression cassette are screened to identify those that show the greatest inhibition of vacuolar processing enzyme expression.

The polynucleotide used for cosuppression may correspond to all or part of the sequence encoding the vacuolar processing enzyme, all or part of the 5′ and/or 3′ untranslated region of a vacuolar processing enzyme transcript, or all or part of both the coding sequence and the untranslated regions of a transcript encoding a vacuolar processing enzyme. In some embodiments where the polynucleotide comprises all or part of the coding region of the vacuolar processing enzyme, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin et al. (2002) The Plant Cell 14: 1417-32. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91: 3490-96; Jorgensen et al. (1996) Plant Molec. Biol. 31: 957-73; Johansen and Carrington (2001) Plant Physiology 126: 930-938; Broin et al. (2002) The Plant Cell 14: 1417-1432; Stoutjesdijk et al (2002) Plant Physiology 129: 1723-1731; Yu et al. (2003) Phytochemistry 63: 753-63; and U.S. Pat. Nos. 5,034,323, 5,283,184, and 5,942,657; each of which is herein incorporated by reference. The efficiency of cosuppression may be increased by including a poly-dT region in the expression cassette at a position 3′ to the sense sequence and 5′ of the polyadenylation signal. See, U.S. Patent Publication 20020048814, herein incorporated by reference.

B. Antisense Suppression

In some embodiments of the invention, inhibition of the expression of a vacuolar processing enzyme may be obtained by antisense suppression. For antisense suppression, the expression cassette is designed to express an RNA molecule complementary to all or part of a messenger RNA encoding a soybean vacuolar processing enzyme. Overexpression of the antisense RNA molecule can result in reduced expression of the native gene. Accordingly, multiple plants lines transformed with the antisense suppression expression cassette are screened to identify those that show the greatest inhibition of vacuolar processing enzyme expression.

The polynucleotide for use in antisense suppression may correspond to all or part of the complement of the sequence encoding the vacuolar processing enzyme, all or part of the complement of the 5′ and/or 3′ untranslated region of a vacuolar processing enzyme transcript, or all or part of the complement of both the coding sequence and the untranslated regions of a transcript encoding a vacuolar processing enzyme. In addition, the antisense polynucleotide may be fully complementary (i.e. 100% identical to the complement of the target sequence) or partially complementary (i.e. less than 100% identical to the complement of the target sequence) to the target sequence. Antisense suppression may be used to inhibit the expression of multiple proteins in the same plant. See, for example, U.S. Pat. No. 5,942,657. Methods for using antisense suppression to inhibit the expression of endogenous genes in plants are described, for example, in Liu et al (2002) Plant Physiology 129: 1732-43 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. Patent Publication 20020048814, herein incorporated by reference.

C. Double Stranded RNA Interference

In some embodiments of the invention, inhibition of the expression of a vacuolar processing enzyme may be obtained by double stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plants lines transformed with the dsRNA interference expression cassette or expression cassettes are then screened to identify plant lines that show the greatest inhibition of vacuolar processing enzyme expression. Methods for using dsRNA interference inhibit the expression of endogenous plant genes are described in Waterhouse et al. (1998) Proc. Natl. Acad. Sci. USA 95: 13959-64, Liu et al. (2002) Plant Physiology 129: 1732-43, and WO publications WO9949029, WO9953050, WO9961631, and WO049035; each of which is herein incorporated by reference.

D. Hairpin RNA Interference and Intron-Containing Hairpin RNA Interference

In some embodiments of the invention, inhibition of the expression of one or more vacuolar processing enzyme may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Gen. 4: 29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97: 4985-90; Stoutjesdijk et al. (2002) Plant Physiology 129: 1723-31; and Waterhouse and Helliwell (2003) Nat. Rev. Gen. 4: 29-38. Methods for using hpRNA interference to inhibit or silence the expression of genes are described, for example, in Chuang and Meyerowitz (2000) Proc. Natl. Acad. Sci. USA 97: 4985-90; Stoutjesdijk et al. (2002) Plant Physiology 129: 1723-31; Waterhouse and Helliwell (2003) Nat. Rev. Gen. 4: 29-38; Pandolfini et al. BMC Biotechnology 3: 7, and U.S. Patent Publication 20030175965, each of which is herein incorporated by reference. A transient assay for the efficiency of hpRNA constructs to silence gene expression in vivo has been described by Panstruga et al. (2003) Mol. Biol. Rep. 30: 135-40, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increase the efficiency of interference. See, for example, Smith et al. (2000) Nature 407: 319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407: 319-320; Wesley et al. (2001) The Plant Journal 27: 581-590; Wang and Waterhouse (2001) Current Opinion in Plant Biology 5: 146-150; Waterhouse and Helliwell (2003) Nat. Rev. Gen. 4: 29-38; Helliwell and Waterhouse (2003) Methods. 30: 289-95, and U.S. Patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, patent publication WO 0200904, herein incorporated by reference.

E. Amplicon-Mediated Interference

Amplicon expression cassettes comprise a plant virus-derived sequence that contains all or part of the target gene, but generally not all of the genes of the native virus. The viral sequences present in the transcription product of the expression cassette allow the transcription product direct its own replication. The transcripts produced by the amplicon may be either sense or antisense relative to the target sequence (i.e. the messenger RNA for a soybean vacuolar processing enzyme). Methods of using amplicons to inhibit the expression of endogenous plant genes are described, for example, in Angell and Baulcombe (1997) EMBO J. 16: 3675-84, Angell and Baulcombe (1999) The Plant Journal 20: 357-362, and U.S. Pat. No. 6,646,805, each of which is herein incorporated by reference.

F. Ribozymes

In some embodiments, the polynucleotide expressed by the expression cassette of the invention is catalytic RNA or ribozyme activity specific for the messenger RNA of a vacuolar processing enzyme. Thus, the polynucleotide causes the degradation of the endogenous messenger RNA, resulting in reduced expression of the vacuolar processing enzyme. This method is described, for example, in U.S. Pat. No. 4,987,071, herein incorporated by reference.

G. Small Interfering RNA or Micro RNA

In some embodiments of the invention, inhibition of the expression of one or more vacuolar processing enzyme may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425: 257-263; herein incorporated by reference.

For miRNA interference, the expression cassette is designed to express an RNA molecule that is modeled on an endogenous miRNA gene. The miRNA gene encodes an RNA that forms a hairpin structure containing a 22 nt sequence that is complementary to another endogenous gene (target sequence). For suppression of VPE expression the 22 nt sequence is selected from a VPE transcript sequence and contains 22 nt of said soybean VPE sequence in sense orientation and 21 nt of an corresponding antisense sequence that is complementary to the sense sequence. miRNA molecules are highly efficient at inhibiting the expression of endogenous genes, and the RNA interference they induce is inherited by subsequent generations of plants.

II. Polypeptides that Inhibit the Expression of Vacuolar Processing Enzymes

In some embodiments, the present invention provides a method for producing a soybean seed storage protein having one or more altered functional properties, where the method comprises the steps of transforming a soybean plant cell with at least one expression cassette comprising a polynucleotide encoding a polypeptide that inhibits the expression of one or more soybean vacuolar processing enzymes, regenerating a transformed plant from the transformed plant cell, and collecting seed from the transformed plant. The polynucleotide may encode any polypeptide that inhibits the expression of a soybean vacuolar processing enzyme.

In one embodiment, the polynucleotide encodes a zinc finger protein that binds to a gene encoding a soybean vacuolar processing enzyme, resulting in reduced expression of the gene. In particular embodiments, the zinc finger protein binds to a regulatory region of a vacuolar processing enzyme gene. In other embodiments, the zinc finger protein binds to a messenger RNA encoding a vacuolar processing enzyme and prevents its translation. Methods of selecting sites for targeting by zinc finger proteins have been described, for example, by U.S. Pat. No. 6,453,242, herein incorporated by reference. Methods for using zinc finger proteins to inhibit the expression of genes in plants are described, for example, in U.S. Patent Publication 20030037355, herein incorporated by reference.

III. Polypeptides that Inhibit the Proteolytic Activity of Vacuolar Processing Enzymes

In some embodiments, the present invention provides a method for producing a soybean seed storage protein having one or more altered functional properties, where the method comprises the steps of transforming a soybean plant cell with at least one expression cassette comprising a polynucleotide encoding a polypeptide that inhibits the proteolytic activity of one or more soybean vacuolar processing enzymes, regenerating a transformed plant from the transformed plant cell, and collecting seed from the transformed plant. The polynucleotide may encode any polypeptide that inhibits the activity of a soybean vacuolar processing enzyme.

In some embodiments of the invention, the polynucleotide encodes an antibody that binds to at least one soybean VPE, and reduces the proteolytic activity of the VPE. In another embodiment, the binding of the antibody results in increased turn-over of the antibody-VPE complex by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21: 35-36, incorporated herein by reference.

In other embodiments of the invention, the polynucleotide encodes a polypeptide that specifically inhibits the proteolytic activity of a soybean vacuolar processing enzyme, i.e. a proteinase inhibitor. In particular embodiments, the proteinase inhibitor is a C-terminal propeptide of a VPE that functions as an auto-inhibitory domain. See, for example, Kuroyangi et al. (2002) Plant Cell Physiol. 43: 143-151, herein incorporated by reference. The expression of other proteinase inhibitors in plant cells is well known in the art. See, for example, Zhong et al. (1999) Molecular Breeding 5: 345-56, herein incorporated by reference.

IV. Methods of Disrupting a Gene Encoding a Soybean Vacuolar Processing Enzyme

In some embodiments of the present invention, the activity of a vacuolar processing enzyme is reduced or eliminated by disrupting the gene encoding the vacuolar processing enzyme. The gene encoding the vacuolar processing enzyme may be disrupted by any method know in the art. For example, in one embodiment the gene is disrupted by transposon tagging. In another embodiment, the gene is disrupted by mutagenizing soybean plants using random or targeted mutagenesis, and selecting for plants that have reduced vacuolar processing enzyme activity.

A. Transposon Tagging

In one embodiment of the invention, transposon tagging is used to reduce or eliminate the proteolytic activity of one or more soybean vacuolar processing enzymes. Transposon tagging comprises inserting a transposon within an endogenous soybean vacuolar processing enzyme gene to reduce or eliminate expression of the vacuolar processing enzyme. By “vacuolar processing enzyme gene” is meant the gene that encodes a soybean vacuolar processing enzyme according to the invention.

In this embodiment, the expression of one or more vacuolar processing enzymes is reduced or eliminated by inserting a transposon within a regulatory region or coding region of the gene encoding the vacuolar processing enzyme A transposon that is within an exon, intron, 5′ or 3′ untranslated sequence, a promoter, or any other regulatory sequence of a soybean vacuolar processing enzyme gene may be used to reduce or eliminate the expression and/or activity of the encoded vacuolar processing enzyme.

Methods for the transposon tagging of specific genes in plants are well known in the art. See, for example, Maes et al. (1999) Trends Plant Sci. 4: 90-96; Dharmapuri and Sonti (1999) FEMS Microbiol. Lett. 179: 53-59; Meissner et al. (2000) Plant J. 22: 265-274; Phogat et al. (2000) J. Biosci. 25: 57-63; Walbot (2000) Curr. Opin. Plant Biol. 2: 103-107; Gai et al. (2000) Nuc. Acids Res. 28: 94-96; Fitzmaurice et al. (1999) Genetics 153: 1919-1928). In addition, the TUSC process for selecting Mu insertions in selected genes has been described in Bensen et al. (1995) Plant Cell 7: 75-84; Mena et al. (1996) Science 274: 1537-1540; and U.S. Pat. No. 5,962,764, each of which is herein incorporated by reference.

B. Mutant Soybean Plants with Reduced Activity for One or More VPEs

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see Ohshima, et al. (1998) Virology 243: 472-481; Okubara et al. (1994) Genetics 137: 867-874; and Quesada et al. (2000) Genetics 154: 421-436; each of which is herein incorporated by reference. In addition, a fast and automatable method for screening for chemically induced mutations, TILLING, (Targeting Induced Local Lesions In Genomes), using denaturing HPLC or selective endonuclease digestion of selected PCR products is also applicable to the instant invention. See McCallum et al. (2000) Nat. Biotechnol. 18: 455-457, herein incorporated by reference.

Mutations that impact gene expression or that interfere with the function (enzymatic activity) of the encoded protein are well known in the art. Insertional mutations in gene exons usually result in null-mutants. Mutations in conserved active site residues are particularly effective in inhibiting the enzymatic activity of the encoded protein. Active site residues of plant VPE's suitable for mutagenesis with the goal to eliminate VPE enzymatic activity have been described. See, for example, Hara-Nishimura, “Asparinyl Endopeptidases” in Handbook of Proteolytic Enzymes, Barrett et al., eds., pp. 746-749 (1998) Academic Press, London; Dalton and Brindley, “Schistosome Legumain” in Handbook of Proteolytic Enzymes, Barrett et al., eds., pp. 749-754 (1998) Academic Press, London; and Chen et al. (1998) FEBS Letters 441:). Such mutants can be isolated according to well-known procedures, and mutations in different VPE loci can be stacked by genetic crossing. See, for example, Gruis et al. (2002) Plant Cell 14: 2863-82.

In another embodiment of this invention, dominant mutants can be used to trigger RNA silencing due to gene inversion and recombination of a duplicated gene locus. See, for example, Kusaba et al. (2003) Plant Cell 15: 1455-67.

The invention encompasses additional methods for reducing or eliminating the activity of one or more vacuolar processing enzymes. Examples of other methods for altering or mutating a genomic nucleotide sequence in a plant are known in the art and, include, but are not limited to, the use of chimeric vectors, chimeric mutational vectors, chimeric repair vectors, mixed-duplex oligonucleotides, self-complementary chimeric oligonucleotides, and recombinogenic oligonucleobases. Such vectors and methods of use, such as, for example, chimeraplasty, are known in the art. Chimeraplasty involves the use of such nucleotide constructs to introduce site-specific changes into the sequence of genomic DNA within an organism. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham et al. (1999) Proc. Natl. Acad. Sci. USA 96: 8774-8778; each of which is herein incorporated by reference.

### Expression Cassettes

The present invention encompasses to the transformation of soybean plants with expression cassettes capable of expressing polynucleotides that reduce or eliminate the proteolytic activity of one or more vacuolar processing enzymes. The expression cassette will include in the 5′-3′ direction of transcription, a transcriptional and translational initiation region (i.e., a promoter) and a polynucleotide of interest, i.e., a polynucleotide capable of directly or indirectly (i.e. via expression of a protein product) reducing or eliminating the activity of one or more soybean vacuolar processing enzymes. The expression cassette may optionally comprise a transcriptional and translational termination region (i.e. termination region) functional in plants. In some embodiments, the expression cassette comprises a selectable marker gene to allow for selection for stable transformants. Expression constructs of the invention may also comprise a leader sequence and/or a sequence allowing for inducible expression of the polynucleotide of interest. See, Guo et al. (2003) Plant J. 34: 383-92 and Chen et al. (2003) Plant J. 36: 731-40 for examples of sequences allowing for inducible expression.

The regulatory sequences of the expression construct will be operably linked to the polynucleotide of interest. By “operably linked” is intended a functional linkage between a promoter and a second sequence wherein the promoter sequence initiates and mediates transcription of the DNA sequence corresponding to the second sequence. Generally, operably linked means that the nucleotide sequences being linked are contiguous.

According to the invention, the proteolytic activity of at least one, at least two, at least three, at least four, at least five, or at least six at least seven, or more than seven vacuolar processing enzymes may be reduced or eliminated in soybean seed. In some embodiments, the polynucleotide of interest is designed to reduce or eliminate the activity of only one vacuolar processing enzyme, while in other embodiments the polynucleotide of interest is designed to inhibit the expression of two or more different soybean vacuolar processing enzymes. Thus in some embodiments, the soybean plants may be transformed with more than one polynucleotide of interest such as at least two polynucleotides of interest, at least three polynucleotides of interest, at least four polynucleotides of interest, at least five polynucleotides of interest, or at least six polynucleotides of interest, at least seven polynucleotides of interest, or more than seven polynucleotides of interest. When two or more polynucleotides of interest are transformed into the same plant cell, they may be expressed from the same expression cassette. Alternatively, the polynucleotides may be comprised in separate expression cassettes.

Various components of the expression constructs of the invention are described below.

A. Promoters

The promoter may be native or analogous or foreign or heterologous to the soybean plant host. Additionally, the promoter may be the natural sequence or alternatively a synthetic sequence. When the promoter is “foreign” or “heterologous” to the plant host, it is intended that the promoter is not the native or naturally occurring promoter for the operably linked sequence encoding the polypeptide of interest The nucleic acids can be combined with constitutive, tissue-preferred, or other promoters for expression in plants. Constitutive promoters include, for example, the core promoter of the Rsyn7 promoter and other constitutive promoters disclosed in WO 99/43838 and U.S. Pat. No. 6,072,050; the core CaMV ³⁵S promoter (Odell et al. (1985) Nature 313: 810-812); rice actin (McElroy et al. (1990) Plant Cell 2: 163-171); ubiquitin (Christensen et al. (1989) Plant Mol. Biol. 12: 619-632 and Christensen et al. (1992) Plant Mol. Biol. 18: 675-689); pEMU (Last et al. (1991) Theor. Appl. Genet. 81: 581-588); MAS (Velten et al. (1984) EMBO J. 3: 2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Tissue-preferred promoters can be utilized to target enhanced expression of the polypeptide of interest within a particular plant tissue. Tissue-preferred promoters include Yamamoto et al. (1997) Plant J. 12(2) 255-265; Kawamata et al. (1997) Plant Cell Physiol. 38(7): 792-803; Hansen et al. (1997) Mol. Gen Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Matsuoka et al. (1993) Proc Natl. Acad. Sci. USA 90(20): 9586-9590; and Guevara-Garcia et al. (1993) Plant J. 4(3): 495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See, Thompson et al. (1989) BioEssays 10: 108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is a preferred endosperm-specific promoter. Glob-1 is a preferred embryo-specific promoter. For dicots, seed-specific promoters include, but are not limited to, bean β-phaseolin, napin, β-conglycinin (see, for example, Kitamura et al. (1984) Theor. Appl. Genet. 68: 253-257, Cho et al. (1989) Nucleic Acids Res. 17: 4386-4389, Kim et al. (1990) Agric. Biol. Chem. 54: 1543-1550, Kim et al. (1990) Protein Engineering 3: 725-731, Jung et al. (1998) Plant Cell 10: 343-357, and Katsube et al. (1998) BBA Gen. Subjects 1379: 107-117, herein incorporated by reference), soybean lectin, cruciferin, and the like.

B. Termination Regions

The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. (1989) Nucleic Acids Res. 17: 7891-7903; and Joshi et al. (1987) Nucleic Acid Res. 15: 9627-9639.

C. Leader Sequences

The expression cassettes may optionally contain 5′ leader sequences in the expression cassette construct. Such leader sequences can act to enhance translation, for example, of a proteinase inhibitor polypeptide of the invention. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86: 6126-6130); potyvirus leaders, for example, TEV leader (Tobacco Etch Virus) (Gallie et al. (1995) Gene 165(2): 233-238), MDMV leader (Maize Dwarf Mosaic Virus), and human immunoglobulin heavy-chain binding protein (BiP) (Macejak et al. (1991) Nature 353: 90-94); untranslated leader from the coat protein mRNA of alfalfa mosaic virus (AMV RNA 4) (Jobling et al. (1987) Nature 325: 622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81: 382-385). See also, Della-Cioppa et al. (1987) Plant Physiol. 84: 965-968. Other methods known to enhance translation can also be utilized, for example, introns, and the like.

D. Selectable Marker Genes

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3: 506-511; Christopherson et al. (1992) Proc. Natl. Acad. Sci. USA 89: 6314-6318; Yao et al. (1992) Cell 71: 63-72; Reznikoff (1992) Mol. Microbiol. 6: 2419-2422; Barkley et al. (1980) in The Operon, pp. 177-220; Hu et al. (1987) Cell 48: 555-566; Brown et al. (1987) Cell 49: 603-612; Figge et al. (1988) Cell 52: 713-722; Deuschle et al. (1989) Proc. Natl. Acad. Aci. USA 86: 5400-5404; Fuerst et al. (1989) Proc. Natl. Acad. Sci. USA 86: 2549-2553; Deuschle et al. (1990) Science 248: 480-483; Gossen (1993) Ph.D. Thesis, University of Heidelberg; Reines et al. (1993) Proc. Natl. Acad. Sci. USA 90: 1917-1921; Labow et al. (1990) Mol. Cell. Biol. 10: 3343-3356; Zambretti et al. (1992) Proc. Natl. Acad. Sci. USA 89: 3952-3956; Baim et al. (1991) Proc. Natl. Acad. Sci. USA 88: 5072-5076; Wyborski et al. (1991) Nucleic Acids Res. 19: 4647-4653; Hillenand-Wissman (1989) Topics Mol. Struc. Biol. 10: 143-162; Degenkolb et al. (1991) Antimicrob. Agents Chemother. 35: 1591-1595; Kleinschnidt et al. (1988) Biochemistry 27: 1094-1104; Bonin (1993) Ph.D. Thesis, University of Heidelberg; Gossen et al. (1992) Proc. Natl. Acad. Sci. USA 89: 5547-5551; Oliva et al. (1992) Antimicrob. Agents Chemother. 36: 913-919; Hlavka et al. (1985) Handbook of Experimental Pharmacology, Vol. 78 (Springer-Verlag, Berlin); Gill et al. (1988) Nature 334: 721-724. Such disclosures are herein incorporated by reference.

The above list of selectable marker genes is not meant to be limiting. Any selectable marker gene can be used in the present invention.

E. Polynucleotides of Interest

Because some of the soybean vacuolar processing enzymes of the invention have high levels of sequence identity in some regions, a polynucleotide of the invention may be designed to reduce or eliminate the activity of one or more vacuolar processing enzymes, for example, by targeting a region of the vacuolar processing enzyme mRNAs that are highly conserved. Alternatively, a polynucleotide may be designed to reduce or eliminate the activity of only one soybean vacuolar processing enzyme. Non-limiting examples of polynucleotides of interest are given below.

1. Sense Sequences

In some embodiments of the invention, inhibition of the expression of a vacuolar processing enzyme may be obtained by cosuppression. For cosuppression, the polynucleotide expressed by the expression constructs corresponds to all or part of an endogenous messenger RNA encoding a soybean vacuolar processing enzyme. The polynucleotide used for cosuppression may correspond to all or part of the messenger RNA encoding the vacuolar processing enzyme, all or part of the 5′ and/or 3′ untranslated region of a vacuolar processing enzyme transcript, or all or part of both the coding sequence and the untranslated regions of a transcript encoding a vacuolar processing enzyme. In some embodiments where the polynucleotide comprises all or part of the coding region of the vacuolar processing enzyme, the expression cassette is designed to eliminate the start codon of the polynucleotide so that no protein product will be transcribed.

The sense sequence typically comprises at least 20 nucleotides, at least 50 nucleotides, at least 75 nucleotides, at least 100 nucleotides, at least 200 nucleotides, at least 500 nucleotides, at least 1000 nucleotides, at least 5000 nucleotides, or more than 5000 nucleotides that correspond to a messenger RNA encoding a soybean vacuolar processing enzyme. The sense sequence generally has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

2. Antisense Sequences

In some embodiments of the invention, inhibition of the expression of a vacuolar processing enzyme may be obtained by antisense suppression. For antisense suppression, the expression cassette is designed to express nucleic molecule or interest corresponding to the complement of all or part of a messenger RNA encoding a soybean vacuolar processing enzyme. The polynucleotide for use in antisense suppression may correspond to all or part of the complement of the sequence encoding the vacuolar processing enzyme, all or part of the complement of the 5′ and/or 3′ untranslated region of a vacuolar processing enzyme transcript, or all or part of the complement of both the coding sequence and the untranslated regions of a transcript encoding a vacuolar processing enzyme.

Thus, antisense sequences are constructed to hybridize with the corresponding mRNA. Modifications of the antisense sequences may be made as long as the sequences hybridize to and interfere with expression of the corresponding mRNA. Thus, antisense sequences may be fully or partially complementary to the target mRNA. In this manner, antisense constructions having 70%, optimally 80%, more optimally 85% sequence identity to the corresponding complements may be used. Furthermore, portions of the antisense nucleotides may be used to disrupt the expression of the target gene. Generally, antisense sequences of at least 20 nucleotides, at least 50 nucleotides, at least 75 nucleotides, at least 100 nucleotides, at least 200 nucleotides, at least 500 nucleotides, at least 1000 nucleotides, at least 5000 nucleotides, or more than 5000 nucleotides of the complement of the target miRNA may be used.

3. Polynucleotides for Double Stranded RNA Interference

In some embodiments of the invention, inhibition of the expression of a vacuolar processing enzyme may be obtained by double stranded RNA (dsRNA) interference. For dsRNA interference, a sense sequence like that described above for cosuppression and an antisense sequence that is complementary to the sense sequence are expressed in the same cell. The antisense sequence may be fully complementary to the sense sequence. Alternatively, the antisense sequence may be partially complementary to the sense sequence so long as it hybridizes to the sense sequence to form a double stranded RNA molecule.

Expression of the sense and antisense molecules can be accomplished by designing the expression cassette to comprise both a sense sequence and a complementary nucleotide sequence. Alternatively, separate expression cassettes may be used for the sense and complementary nucleotide sequences.

4. Polynucleotides for hpRNA Interference and ihpRNA Interference

In some embodiments of the invention, inhibition of the expression of one or more vacuolar processing enzyme may be obtained by hairpin RNA (hpRNA) interference or intron-containing hairpin RNA (ihpRNA) interference. For hpRNA interference, the expression cassette is designed to express nucleic molecule of interest that hybridizes with itself to form a hairpin structure that comprises a single-stranded loop region and a base-paired stem. In some embodiments, the base-paired stem region is formed by hybridization between a sense sequence corresponding to all or a portion of a messenger RNA encoding a vacuolar processing enzyme and an antisense sequence that is complementary to the sense sequence. In other embodiments, the base-paired stem region is formed by hybridization between two sequences that are unrelated to an endogenous messenger RNA, and the loop region comprises all or part of the messenger RNA sequence for a soybean vacuolar processing enzyme.

Thus, in some embodiments, the sense sequence comprises at least 19, at least 30, at least 50, at least 100, at least 500, at least 1000, or more than 100 nucleotides corresponding to the mRNA encoding a soybean vacuolar processing enzyme (i.e. the target mRNA). The sense sequence generally shares at least 94% or more sequence identity with the corresponding region of the target mRNA, such as, for example, at least 95% or more sequence identity, at least 96% or more sequence identity, at least 97% or more sequence identity, at least 98% or more sequence identity, or at least 99% or more sequence identity. The antisense sequence may be fully complementary to the sense sequence. Alternatively, the antisense sequence may be partially complementary to the sense sequence so long as it hybridizes to the sense sequence to form a stem region. The hpRNA polynucleotide additional comprises a spacer or loop sequence operably 3′ of the sense sequence and 5′ of the antisense sequence. When the spacer sequence does not contain an intron, it is generally preferred to make the loop sequence as short as possible while still providing enough of a loop to allow the sense sequence to hybridize with the antisense sequence. Accordingly, the loop sequence is generally less than 1000 nucleotides, less than 900 nucleotides, less than 800 nucleotides, less than 700 nucleotides, less than 600 nucleotides, less than 500 nucleotides, less than 400 nucleotides, less than 300 nucleotides, less than 200 nucleotides, less than 100 nucleotides, or less than 50 nucleotides.

In other embodiments, the base paired stem structure is formed by the hybridization of a sense sequence that does not correspond to an endogenous sequence found in the host soybean plant, and an antisense sequence complementary to the sense sequence. The sense and antisense sequences flank a loop region that comprises all or part of a sequence corresponding to a messenger RNA encoding a soybean vacuolar processing enzyme. Generally, the sense and antisense sequences will each be at least 40-50 nucleotides in length, such as 50-100 nucleotides in length, or 100-300 nucleotides in length. See, WO 0200904 for examples of sense and antisense sequences that may be used. The loop sequence corresponding to a messenger RNA encoding a soybean vacuolar processing enzyme generally comprises at least 25 nucleotides corresponding to the messenger RNA encoding the soybean vacuolar processing enzyme, and may comprise at least 50 nucleotides, at least 100 nucleotides, at least 200 nucleotides, or at least 300 nucleotides in length. The loop sequence generally shares at least 80% sequence identity with a messenger RNA encoding a soybean vacuolar processing enzyme, and may share at least 85% sequence identity, at least 90% sequence identity, or at least 95% sequence identity with a messenger RNA encoding a soybean vacuolar processing enzyme.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increase the efficiency of interference. Any intron that is spliced in soybean may be used according to the invention. Non-limiting examples of introns that may be used include the orthophosphate dikinase 2 intron 2 (pdk2 intron) described in U.S. Patent publication No. 20030180945, the catalase intron from Castor bean (Accession number AF274974), the Delta12 desaturase (Fad2) intron from cotton (Accession number AF331163), the Delta 12 desaturase (Fad2) intron from Arabidopsis (Accession number AC069473), the Ubiquitin intron from maize (Accession number S94464), and the actin intron from rice.

Transformation and Regeneration

In some embodiments, the methods of the invention comprise the steps of transforming and regenerating soybean plants. Suitable methods of introducing nucleotide sequences into plant cells and subsequent insertion into the plant genome include microinjection (Crossway et al. (1986) Biotechniques 4: 320-334), electroporation (Riggs et al. (1986) Proc. Natl. Acad. Sci. USA 83: 5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3: 2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6: 923-926) and Lec1 transformation (WO 00/28058). Also see Weissinger et al. (1988) Ann. Rev. Genet. 22: 421-477; Sanford et al. (1987) Particulate Science and Technology 5: 27-37 (onion); Christou et al. (1988) Plant Physiol. 87: 671-674 (soybean); McCabe et al. (1988) Bio/Technology 6: 923-926 (soybean); Finer and McMullen (1991) In Vitro Cell Dev. Biol. 27P: 175-182 (soybean); Singh et al. (1998) Theor. Appl. Genet. 96: 319-324 (soybean); Datta et al. (1990) Biotechnology 8: 736-740 (rice); Klein et al. (1988) Proc. Natl. Acad. Sci. USA 85: 4305-4309 (maize); Klein et al. (1988) Biotechnology 6: 559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91: 440-444 (maize); Fromm et al. (1990) Biotechnology 8: 833-839 (maize); Hooykaas-Van Slogteren et al. (1984) Nature (London) 311: 763-764; Bowen et al., U.S. Pat. No. 5,736,369 (cereals); Bytebier et al. (1987) Proc. Natl. Acad. Sci. USA 84: 5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9: 415-418 and Kaeppler et al. (1992) Theor. Appl. Genet. 84: 560-566 (whisker-mediated transformation); D'Halluin et al. (1992) Plant Cell 4: 1495-1505 (electroporation); Li et al. (1993) Plant Cell Reports 12: 250-255 and Christou and Ford (1995) Annals of Botany 75: 407-413 (rice); Osjoda et al. (1996) Nature Biotechnology 14: 745-750 (maize via Agrobacterium tumefaciens); all of which are herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5: 81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

### Plants and Seed

The invention also provides soybean plants that are genetically modified or mutagenized to reduce or eliminate the activity of one or more vacuolar processing enzymes in seed, and transformed seed of these plants. The term “genetically modified” as used herein refers to a plant cell or plant that is modified in its genetic information by the introduction of one or more foreign polynucleotides, and that the expression of the foreign polynucleotides leads to a phenotypic change in the plant. By “phenotypic change,” it is intended a measurable change in one or more cell functions. For example, the genetically modified plants of the present invention show reduced or eliminated expression or enzymatic activity of one or more vacuolar processing enzymes. Also provided are soybean plants that have been mutagenized and carry a mutation in one or more genes encoding a vacuolar processing enzyme that results in reduced activity of the encoded vacuolar processing enzyme.

The soybean plants encompassed by the invention may be genetically modified or mutated to inhibit the expression or enzymatic activity of at least one, at least two, at least three, at least four, at least five, at least six, or at least seven or more vacuolar processing enzymes. Those of ordinary skill in the art recognize that this can be accomplished in any one of a number of ways. For example, each of the expression cassettes for inhibiting the expression or enzymatic activity of the vacuolar processing enzymes can be operably linked to a promoter and then joined together in a single continuous fragment of DNA comprising an expression cassette. Such an expression cassette can be used to transform a plant to produce the desired outcome. Alternatively, separate plants can be transformed with expression cassettes capable of expressing a polynucleotide, which inhibits the expression of different vacuolar processing enzyme. A single plant that is genetically modified to inhibit the expression or the enzymatic activity of two or more vacuolar processing enzymes can then be produced by transforming a selected genetically modified plant to inhibit the expression of a different vacuolar processing enzyme, and selecting for plants showing inhibition in expression or enzymatic activity of multiple vacuolar processing enzymes. Multiple rounds of transformation and selection may be required to produce the desired plant.

Alternatively, a single plant that is genetically modified or mutagenized to inhibit the expression or the enzymatic activity of two or more vacuolar processing enzymes can be produced through one or more rounds of cross pollination utilizing the previously selected seed-protease deficient plants as parents. Methods for cross pollinating plants are well known to those skilled in the art, and are generally accomplished by allowing the pollen of one plant, the pollen donor, to pollinate a flower of a second plant, the pollen recipient, and then allowing the fertilized eggs in the pollinated flower to mature into seeds. Progeny containing the entire complement of heterologous coding sequences of the two parental plants can be selected from all of the progeny by standard methods available in the art as described supra for selecting transformed plants. If necessary, the selected progeny can be used as either the pollen donor or pollen recipient in a subsequent cross pollination.

### Methods of Determining % Sequence Identity

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4: 11-17; the local alignment algorithm of Smith et al. (1981) Adv. Appl. Math. 2: 482; the global alignment algorithm of Needleman and Wunsch (1970) J. Mol. Biol. 48: 443-453; the search-for-local alignment method of Pearson and Lipman (1988) Proc. Natl. Acad. Sci. USA 85: 2444-2448; the algorithm of Karlin and Altschul (1990) Proc. Natl. Acad. Sci. USA 872264, modified as in Karlin and Altschul (1993) Proc. Natl. Acad. Sci. USA 90: 5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 10 (available from Accelrys Inc., 9685 Scranton Road, San Diego, Calif. USA). Alignments using these programs can be performed using the default parameters. The CLUSTAL program is well described by Higgins et al. (1988) Gene 73: 237-244 (1988); Higgins et al. (1989) CABIOS 5: 151-153; Corpet et al. (1988) Nucleic Acids Res. 16: 10881-90; Huang et al. (1992) CABIOS 8: 155-65; and Pearson et al. (1994) Meth. Mol. Biol. 24: 307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215: 403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a polypeptide of the invention. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.hlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89: 10915).

### Experimental

Altered Solubility Profile for Arabidopsis thaliana Seed Storage Proteins in the Absence of Vacuolar Processing Enzyme Activity

I. Methods

A. Isolation of the αype::dSpm1 Allele

A putative dSpm transposon insertion in αype was identified in DNA of SLAT (Sainsbury Laboratory Arabidopsis thaliana dSpm Transposants) pool 5.38 by probing a filter blot, obtained from the Sainsbury Laboratory displaying flanking DNA of the Sainsbury dSpm transposon insertion population, with a genomic DNA probe corresponding to the αype gene.

Confirmation and localization of the dSpm insertion within αype (αype::dSpm1 allele) was accomplished by PCR of pool 5.38 genomic DNA (obtained from the Sainsbury laboratory), PCR product isolation, and DNA sequencing as previously described. Plants homozygous for the αype::dSpm 1 allele were identified by PCR from progeny of the 5.38 seed pool. Homozygosity was confirmed by the lack of PCR detectable wild-type alleles in the F2 progeny following self-pollination of putative αype::dSpm 1 homozygous plants.

B. Isolation of the γvpe::T-DNA1 Allele

The SIGnAL (Salk Institute Genomic Analysis Laboratory) database (available at signal.salk.edu/cgi-bin/tdnaexpress) of T-DNA left border adjacent sequences was queried with the γVPE sequence to identify a seed stock (Salk_{—}010372) containing an insertion within the 5^{th} exon of γVPE. Seeds from this line were obtained from the Arabidopsis Biological Resource Center (ABRC), and seedlings screened by PCR to identify plants homozygous for the γVPE::T-DNA 1 allele. DNA was isolated with the DNeasy Plant Mini kit (Qiagen, Inc., Valencia, Calif.) according to the manufacturer's protocol and subjected to PCR to detect the γvpe::T-DNA 1 allele. Homozygous γVPE::T-DNA plants were confirmed by the lack of PCR detectable wild-type alleles in the F2 progeny following self-pollination.

C. Genetic Stacking and PCR Identification of Homozygous Mutants

Genetic stacking and isolation of VPE mutant plants was performed as follows. First, plants homozygous for both the βvpe::dSpm1 and δvpe::dSpm1 alleles (Gruis et al. (2002) Plant Cell 14: 2863-82) were crossed with plants homozygous for αvpe::dSpm1. Second, plants among the segregating F2 progeny (following F1 self pollination) identified as homozygous for αvpe::dSpm1, βvpe::dSpm1 and εvpe::dSpm1 were then crossed with plants homozygous for γVPE::T-DNA1. For PCR screening of F2 progeny following F1 self pollination of the second cross, DNA was prepared from one rosette leaf of each plant prior to flowering. Fresh tissue was harvested into 1.1 ml minitubes of a 96-well Megatiter-Plate (Biological Band Continental Lab Products) on ice. A 5/32″ steel bead and 200 μl of extraction buffer (10% w/v potassium ethyl xanthogenate, 100 mM Tris pH 7.5, 2 M NaCl and 10 mM EDTA) were added to each sample immediately prior to homogenization in a Raptor/Geno/Grinder (Spex CentiPrep Inc., Metuchen, N.J.) for 1 minute at 7000 strokes/minute. Following incubation at 65° C. for 30 minutes, the samples were cooled on ice for 15 minutes, centrifuged at 3,000 g for 15 minutes and 150 μl of supernatant transferred to a new tube. A second centrifugation was again performed to remove debris and 100 μl of supernatant was transferred to a new tube containing 150 μl of ice cold 2-propanol. The DNA-precipitate was pelleted by centrifugation, rinsed with 300 μl of cold 70% ethanol v/v, dried for 20 minutes in a 65° C. air incubator and incubated at 65° C. for 20 minutes with 150 μl of 5 mM Tris-HCl pH 8.0. 3 μl of this DNA preparation were used per PCR reaction. The putative genotypes of selected plants of interest identified from the initial large scale screen were then confirmed by a second round of PCR analysis using DNA isolated from an independently harvested rosette leaf with the DNeasy Plant Mini kit (Qiagen, Inc., Valencia, Calif.) according to the manufacturer's protocol. Homozygosity of the various mutant allele combinations was confirmed by the lack of detectable wild-type alleles in the F3 progeny following self-pollination.

D. γVPE Knock-Down/βvpe Plants

Confirmation of the γVPE null-allele phenotype was accomplished by transforming βvpe mutant plants with an intron-spliced self-complimentary hairpin RNAi construct (Smith et al. (2000) Nature 407: 319-320) designed to knock down γVPE expression. The RNAi portion of the vector was constructed using standard cloning techniques to splice the β phaseolin promoter described by Slightom et al., Custom polymerase chain reaction engineering plant expression vectors and genes for plant expression, pp. 1-55 in Plant Molecular Biology Manual, Gelvin and Schilperoort, eds., Dordrect:Kluwer Academic Publishers (1991), with an rtPCR-amplified 500 bp fragment (nucleotides 27-526 of NCBI Accession No. AF370160) of γVPE in the sense orientation, a 1133 bp PCR-amplified FAD2 intron sequence (nucleotides 142-1274 of NCBI Accession No. AC069473), and a 500 bp fragment of γVPE in reverse orientation. The transformation vector also contained the constitutive promoter SCP1 described by U.S. Pat. No. 6,555,673 to Bowen et al. to drive expression of the selectable marker, the neomycin phosphotransferase II gene. Agrobacterium-mediated transformation using strain GV3101 carrying the helper plasmid pMP90 was performed using the flora dip method described by Clough and Bent (1998) Plant J. 16: 735-43). Kanamycin resistant seedlings were selected, allowed to self-pollinate, and T1 seed of γVPE knock-down events were analyzed by SDS-PAGE.

E. SDS-PAGE and Immunoblotting

Developing, germinating and mature seed were collected and protein was extracted under reducing conditions as described by Gruis et al. (2002) Plant Cell 14: 2863-82. Protein extraction for SDS-PAGE under oxidizing conditions was accomplished by homogenization of mature seed meal with a 20-fold v/w excess of ice-cold 2% SDS, 50 mM Tris-HCl pH 6.8, and 100 mM iodoacetamide. Samples were incubated on ice, for 5 minutes at room temperature, and finally for 5 minutes at 100° C. After incubation, the samples were treated as reduced protein extracts as described in Gruis et al. (2002) Plant Cell 14: 2863-82, except that DTT was omitted from SDS-PAGE sample buffer. Proteins were electrophoretically separated by SDS-PAGE using one of the following methods: Tris-Tricine gels (8% spacer and 15% separating), Tris-Tricine gels using a 8% spacer and a 12% separating gel or Tris-Glycine 4-20% gradient mini-gels (BioRad, Hercules, Calif.). Immunoblotting was performed using either a 1:2500 dilution of anti-sera generated using rape seed cruciferin to detect legumin-type globulins or a 1:5000 dilution of anti-sera generated using HPLC-purified Arabidopsis napin-type albumins. The legumin-type globulin anti-sera cross reacts with α-chain epitopes of Arabidopsis legumin-type globulins and the Arabidopsis napin-type albumin specifically detects epitopes on the large chains.

F. Linear Sucrose Density Gradient Separations

Dry mature seed was ground at room temperature using a porcelain mortar and pestle and 25 mg of the resulting meal was defatted in 2 ml microcentrifuge tubes by three sequential 1 ml hexane extractions at room temperature. Following vacuum desiccation, the meal was re-suspended in 20 v/w ice cold extraction buffer (100 mM sodium phosphate buffer pH 7, 400 mM KCl) containing 1 mM Pefabloc (Roche Molecular Biochemicals, Indianapolis, Ind.) and incubated at 4° C. for 40 minutes with constant agitation. The supernatant was then recovered following a 10 min centrifugation at 20,800 g and the protein concentration was determined using the bicinchoninic acid (BCA) Protein Quantitation Assay (Pierce, Rockford, Ill.) standardized using bovine serum albumin (Pierce, Rockford, Ill.). Following extraction, protein samples were immediately loaded onto sucrose density gradients.

Linear sucrose density gradients (6-20%) were prepared in SW40 ultracentrifuge tubes (Beckman Coulter Instruments Inc., Fullerton, Calif.) using the BIOCOMP Gradient Maker 107 ip (BioComp Instruments Inc., New Brunswick, Canada) per the manufacturer's instructions. 200 μl of protein extract (˜1.5 mg of protein) was applied to the top of the prepared gradients. Proteins were then fractionated by centrifugation at 37,000 rpm (SW40 rotor) at 4° C. for 21 hours. Following centrifugation, gradients were fractionated using a BIOCOMP Piston Gradient Fractionator-151 (BioComp Instruments Inc., New Brunswick, Canada) at 0.3 mm/sec and collected using a Frac-200 fraction collector (Pharmacia LKB, Uppsala, Sweden) set up to collect 12 drops (˜300 μl) per fraction. Any potential pellet remaining at the bottom of the tube was re-suspended in 100 μl of SDS protein extraction buffer for analysis. The protein quantity in each gradient fraction was determined using the BCA assay (Pierce, Rockford, Ill.) and results plotted for each fraction as a percentage of the protein detected in all fractions. Proteins of known sedimentation coefficients; chymotrypsin (2.6S), bovine serum albumin (4.4S), aldolase (7.3S) and catalase (11.3S) (Pharmacia LKB, Uppsala, Sweden) were separated in parallel gradients and used as a reference to assign sedimentation coefficients to the Arabidopsis seed protein gradient fractions.

Prior to analysis by SDS-PAGE each gradient fraction sample was concentrated 5 fold using Micron YM-3 centrifugal filter devices (Millipore, Bedford, Mass.). For Coomassie Brilliant Blue R-250 stained SDS-PAGE analysis, 10 μl of sample was incubated at 100° C. for 5 minutes with 4 μl of SDS-PAGE loading buffer (250 mM Tris pH 6.8, 500 mM DTT, 10% w/v SDS, 0.5% w/v bromophenol blue, 50% v/v glycerol). Samples were then electrophoresed in 26-well 4-20% gradient Tris-HCl mini-gels (BioRad, Hercules, Calif.). Immunoblotting was carried out as described using 2 μl of each sample.

G. Solubility Profiling

Proteins were extracted and separated using linear sucrose density gradients (see above). Legumin-type globulin protein from wild-type seed was obtained by pooling fractions #24-30 from 4 parallel linear sucrose density gradient separations of wilt-type seed proteins. Legumin-type globulin protein from vpe-quad mutant seed was obtained by pooling fractions # 15-21 from 4 parallel linear sucrose density gradients of vpe-quad seed proteins. Proteins contained in these pooled fractions were first subjected to a 1500 fold dilution into buffer (150 mM NaCl, 20 mM Tris pH 8.0) and subsequently concentrated to ˜20 mg/ml using Amicon Ultra 10,100 MWCO centrifugal filter devices (Millipore, Bedford, Mass.) according to manufacturer's instructions. Following this procedure each protein sample was quantified and adjusted to a final concentration of 14 mg/ml using the BCA assay (Pierce, Rockford, Ill.). For each sample (12S wild-type and 9S quad) dilutions of protein into several pH buffers (Na Acetate-acetic acid, pH 3.5, pH 4.0, pH 4.5, pH 5.5; MES-NaOH pH 5.5, pH 6.0, pH 6.5; Hepes-HCl, pH 7.0, pH 7.5, pH 8.0; Tris-HCl pH 8.5) was performed at room temperature. Each pH condition was set up as a 30 μl reaction mixture in a microcentrifuge tube containing a final concentration of 25 mM buffer, 10 mM NaCl and 0.9 mg/ml protein. Following incubation at room temperature for 2 hours, samples were subjected to centrifugation at 20,800 g for 10 min. Supernatants were then assayed for protein content using the BCA assay (Pierce, Rockford, Ill.) and results for each sample plotted as a percentage of protein remaining in the supernatant (soluble).

II. Results

A. Detection of Vegetative-Type VPE Gene Expression in Developing Seed

Because vegetative-type VPE gene expression is induced in vegetative-tissues under stress conditions (Kinoshita et al. (1999) Plant J. 19: 43-53), the possibility that vegetative-type VPE gene expression may be induced due to abnormal accumulation of precursor proteins in βvpe mutant seed was tested. Semi-quantitative multiplexed RT-PCR was performed using γVPE specific primers in combination with primers specific for a constitutively expressed transcript (cytosolic ribosomal protein S11). This analysis detected γVPE transcript in a vegetative control sample (leaf), known to express γVPE. However, contrary to expectations, prominent γVPE-specific amplification products were also detected in developing seed of wild-type plants. The ratio of the intensity of the γVPE-specific band compared to the S11-specific band indicated similar amounts of γVPE transcript were present in leaf and developing seed samples of wild-type and βVPE/εVPE double mutants. To confirm and quantify γVPE transcript in developing seed, quantitative real-time PCR was performed using independently isolated RNA from developing seed of both wild-type and the βvpe/εvpe double mutants. This analysis also detected γVPE transcript in developing wild-type seed and showed no significant change of γVPE transcript level in the mutant sample.

To further substantiate this observation and to relate the quantity and/or significance of γVPE expression in seed to the other members of the VPE gene family, queries of several Arabidopsis Massively Parallel Signature Sequencing (MPSS) high-resolution gene expression datasets with conceptual MPSS expressed sequence tags (ESTs) of Arabidopsis VPE genes were performed. MPSS gene expression datasets are essentially EST sequencing experiments each consisting of 1 to 2 million independently derived MPSS ESTs from a single tissue source. Therefore, these very deep EST sequence libraries provide quantitative gene expression data reported in parts per million (ppm) for each transcript. Corroborating the RT-PCR results, γVPE transcripts are present in developing seed concurrently with βVPE and δVPE transcripts. Moreover, the second Arabidopsis vegetative-type VPE gene, αVPE, is also expressed in developing seed, albeit at much lower levels (4-10-fold less) than γVPE. The βVPE expression profile is similar to the expression profile of seed storage protein genes, showing peak expression in seed 14 days after anthesis. At this stage, βVPE is the most prominent VPE gene transcript detected, approximately 3-fold more prevalent than γVPE transcript. γVPE transcript is the second most abundant VPE gene transcript detected at this stage (MSS), however, 2-3 fold higher levels of this transcript are detected earlier during seed development. γVPE is also the only VPE gene for which significant levels of transcript are detected in vegetative tissues including leaves and roots. The δVPE gene is the most abundant VPE gene transcript during the cell division stage of seed development and in germinating seed. δVPE transcript is also present at significant levels in all other developing seed stages assayed. Together, these data indicate that all four Arabidopsis VPE genes, including vegetative-type VPE family members, are significantly expressed in developing seed during storage protein accumulation.

B. Isolation of Vegetative-Type VPE Gene Knock-Out Mutants

To investigate a potential function of the two Arabidopsis vegetative-type VPE genes during seed development, plants containing DNA insertion alleles in the αype and γVPE genes were isolated. A putative dSpm transposon insertion allele of αype (αvpe::dSpm1) was identified in pool 5.38 of the Sainsbury Laboratory collection by reverse screening using SLAT blots probed with DNA of α-VPE. DNA flanking the insertion site was cloned and sequenced to determine the location of the dSpm element within the gene. The dSpm insertion in αype::dSpm1 is located 249 bp downstream of the translational start codon in the intron following the first exon of the gene. The dSpm element used in creating the Sainsbury mutant collection has been designed to contain transcriptional stop sites in either orientation such that intronic insertion events would interfere with gene transcription. To test whether αype::dSpm1 is a knock-out allele, multiplexed RT-PCR using αVPE-specific primers annealing downstream of the dSpm insertion site in combination with primers specific for a control transcript (cytosolic ribosomal protein S11) was performed with RNA isolated from 14 DAA seed of two homozygous αype::dSpm1 plants and from two wild-type plants. A PCR product corresponding to αype transcript was amplified only in wild-type seed samples and not in samples of seed homozygous for the αype::dSpm1 allele, classifying the αvpe::dSpm1 allele as a null-allele.

A putative T-DNA insertion allele of γVPE (γVPE::T-DNA1) was identified by querying the SIGnAL website (available at salk.edu). Seed from the corresponding mutant line (Salk_{—}010372) was obtained from the Arabidopsis Biological Resource Center and plants homozygous for the γVPE::T-DNA1 allele were subsequently identified using allele specific PCR. Analysis of the T-DNA adjacent DNA sequence was used to identify the T-DNA integration site as located within exon 5 of the γVPE gene. To test whether γVPE::T-DNA1 is a null allele, RT-PCR was performed essentially as described above for αype::dSpm1. γVPE transcript was clearly detected in wild-type control plants but not in homozygous γVPE::T-DNA1 plants, a result indicative of a knock-out allele.

Mutants homozygous for either αype::dSpm1 or γVPE::T-DNA1 were examined for visible phenotypes under normal growth conditions. No effects were observed on germination rate, vegetative growth rate, plant architecture, seed set, or senescence compared to wild-type controls. Moreover, no differences between protein profiles of mutant and wild-type seed were detected.

C. Genetic Stacking of VPE Mutant Alleles

Genetic stacking of null-alleles of the four unlinked Arabidopsis VPE genes was performed. A βvpe/δvpe double mutant was first crossed to the αvpe mutant and triple mutant plants (αvpe/βvpe/δvpe), homozygous for the respective null-alleles at each locus, were identified by allele-specific PCR analysis of the segregating F2 progeny following F1 self-pollination. The αype/βvpe/δvpe triple mutant was then crossed to the γVPE mutant and, after F1 self-pollination, a total of 1132 F2 progeny plants were screened for the absence and presence of wild-type and mutant alleles at each VPE locus. This screen identified two αvpe/βvpe/γvpe/δvpe quadruple-mutant plants (referred to herein as vpe-quad) homozygous for null-alleles at all four VPE loci, as well as plants with all possible combinations of homozygous triple-mutant alleles and homozygous double mutant alleles of VPE genes. A minimum of two plants of each genotype was isolated (not all data shown). Progeny of these plants, including vpe-quad plants, were grown for two generations under normal growth conditions side-by-side with wild-type plants and closely inspected for any phenotypic variation compared to the wild-type controls. In all cases, no effects were observed on germination rate, vegetative growth, flowering time, seed set, senescence, plant architecture or light-microscopic seed morphology.

D. Seed Protein Profiles of VPE Mutants

The impact of removal of VPE expression on seed storage protein processing was examined with seed protein extracts (FIG. 1) from plants with the mutant allele combinations described in the description of the figure. A minimum of two plants of each genotype were analyzed to ensure that SDS-PAGE protein profiles shown in FIG. 1 are representative for each investigated genotype. Several observations can be made from this gel analysis. The double null-mutant of the vegetative-type VPE genes (αype/γVPE) does not detectably alter seed protein processing. Mutants of seed-type VPEs, either βvpe or βvpe/δvpe double mutants, show subtle changes in the mature seed protein profiles. The combination of the βvpe/δvpe double mutants with the vegetative-type αvpe mutant (αvpe/βvpe/δvpe) do not result in any discernable additional change in the protein profile beyond what is observed for the seed-type VPE mutants alone. However, dramatic differences in protein profiles are observed in seeds of plants that are homozygous for null-alleles at both the βvpe loci and γVPE loci. The accumulation of polypeptides of the apparent molecular mass predicted for pro-protein forms of the legumin-type globulin proteins is increased while polypeptides corresponding to mature α- and β-chains are significantly decreased. Additionally, accumulation of the mature small chains of napin-type albumins is decreased and polypeptides of apparent molecular mass greater than that observed for mature large chains significantly accumulate. Interestingly, the comparison of the protein of the βvpe/γVPE/δvpe mutants with the protein profile of vpe-quad mutants reveals subtle additional changes of legumin-type globulin and napin-type albumin accumulation that can be attributed to the αype null-allele. Therefore, both vegetative-type VPEs are involved in seed protein processing.

To independently corroborate the observed null-allele phenotype of vegetative-type VPEs, a βvpe mutant plant was transformed with a RNA silencing construct to suppress γVPE expression. The seed protein profile from a resulting γVPE knock-down/βvpe plant is similar to that observed for βvpe/γVPE/εvpe triple mutants supporting the conclusion that the observed seed protein profile phenotypes of the vegetative-type VPE mutants are indeed a direct result of the insertional interruption of VPE genes.

E. Alternative Proteolytic Processing of Seed Proteins

In addition to detecting polypeptides of an apparent molecular mass consistent with pro-forms of legumin-type globulins, several novel polypeptides of lesser molecular masses were observed in vpe-quad seed under reducing SDS-PAGE conditions. At least some of these polypeptides cross-reacted with α-chain specific legumin antibodies identifying them as alternatively processed legumin-type globulin polypeptides containing α-chain epitopes. To determine if any of the other novel polypeptides are disulfide-linked to these legumin α-chain-related polypeptides, seed proteins were extracted in the presence of iodoacetamide (IAA) and separated by SDS-PAGE under oxidizing conditions. Alkylation of free sulfhydryl groups with IAA was necessary to prevent disulfide interchange reactions in legumin-type globulin subunits. Without IAA added, even under oxidizing conditions, these reactions caused extensive breakage of disulfide-bonds between α- and β-chains of Arabidopsis legumin-type globulins. As expected, under oxidizing SDS-PAGE conditions, wild-type seed protein bands shifted to apparent molecular masses consistent with legumin-type pro-globulins (˜50 kD) and napin-type pro-albumins (˜12 kD), indicative of disulfide linked chains for each class of storage proteins. When IAA-treated protein from the vpe-quad seed was analyzed, it was likewise evident that many of the novel polypeptides observed under reducing SDS-PAGE conditions were size-shifted under oxidizing conditions. Most polypeptides appeared to migrate at sizes similar to pro-proteins, including the bands that corresponded to legumin-type globulin polypeptides with α-chain epitopes. However, at least one of these legumin-specific bands (˜40 kD) appears to be smaller than legumin-type pro-globulins, indicating alternative cleavage that results in the loss of a polypeptide chain (˜10 kD), which is not disulfide-linked to the alternatively processed subunit. Additionally in vpe-quad seed, napin-type albumins, size shifted under oxidizing conditions, are slightly greater in apparent molecular mass than the napin-type polypeptides accumulated in wild-type. This observation is consistent with efficient VPE-independent cleavage of napin-type pro-polypeptides into disulfide linked large and small chains that contain additional amino acids.

F. N-Terminal Amino Acid Sequence Analysis

To further investigate the nature of alternative processing in developing vpe-quad seed, Edman degradation was performed for several prominent polypeptide bands that appeared to be novel compared to wild-type. Separation of seed proteins using linear sucrose density gradients and SDS-PAGE was used to further enrich protein bands prior to sequencing. All polypeptides successfully identified from the vpe-quad 9S and 2S fractions were derivatives of legumin-type globulins and napin-type albumins respectively. The majority of identifications corresponded to the two most highly expressed seed storage protein genes, legumin-type globulin cruciferin 1 and napin-type albumin 3.

Six polypeptides were successfully sequenced and identified from the 9S fraction of vpe-quad. The N-terminal sequence of two polypeptides with an apparent molecular mass consistent with pro-forms of legumin-type globulins, each corresponded to the sequence of a different legumin-type globulin immediately downstream of the predicted signal peptide. Therefore, sequence and molecular mass identify these two legumin-type globulin proteins as unprocessed precursors.

Instead of mature β-chains of legumin-type globulins, vpe-quad seed accumulated prominent polypeptides that are approximately 1 kD greater in molecular mass than β-chains accumulated in wild-type seed. Similar to wild-type β-chains, these proteins failed to bind α-chain specific legumin anti-sera. The N-terminal sequence obtained for one of these polypeptides corresponded to the hyper-variable region sequence of a legumin-type globulin, 11 residues upstream of the Asn-Gly polypeptide bond that is normally cleaved in wild-type seed by VPE. A second polypeptide matched the N-terminal sequence immediately downstream of the signal peptide. However, the apparent mass of this polypeptide was ˜32 kD, which is 1-2 kD less than the calculated mass for the mature α-chain derived from this protein. The sizes and sequences of the polypeptides with band ID 6 and 10 are therefore consistent with the same alternative cleavage event occurring in the hyper-variable region of the legumin-type globulin, upstream of the normally processed Asn-Gly bond.

In addition to proteolytic cleavage of legumin-type globulins yielding novel α- and β-chain-like fragments, other fragments of lesser molecular mass than either α- or β-chains were also identified. Several polypeptides that were all derived from a single legumin-type globulin gene were identified, indicating that no single preferred alternative-processing pathway appeared to exist to compensate for the lack of VPE activity. N-terminal amino acid sequencing of napin-type albumin polypeptides isolated from vpe-quad seed allowed for the successful identification of most of these polypeptides. The vast majority of napin-type albumin did not accumulate as a precursor-like form, but is instead processed to novel forms.

All cleavage sites of napin-type albumins so far identified by amino-terminal sequencing in vpe-quad seed involved a Phe residue at the P1 or P1′ position. Additionally, the cleavage of at least one legumin-type polypeptide also occurred at a Phe in P1′. Proteolysis at these locations is consistent in sequence context with cleavage by a member(s) of the aspartic protease gene family.

G. Impact of Processing on Legumin-Type Globulin Solubility

The solubility profile of legumin-type globulins changes following VPE-specific processing of pro-forms into mature α- and β-chains such that a profound decrease in solubility under acidic conditions (pH 4.5-5.5) is observed. To determine if legumin-type globulin accumulated in vpe-quad seed shares similar solubility properties with wild-type VPE-processed protein, the solubility profile of the wild-type 12S proteins was compared to the 9S proteins of vpe-quad (FIG. 2). The solubility profile of VPE-processed legumin-type globulin (wild-type) shows the protein to be largely soluble at pH 7-8.5 and 3.5-4. At intermediate pH ranges, the solubility of the wild-type protein fraction is gradually reduced with the majority of protein being insoluble at pH 5.5-6.0. Contrasting this result, the solubility profile of legumin-type globulin accumulated in vpe-quad seed shows the protein to be mostly soluble at pH 7.5-8.5, and mostly insoluble at pH 3.5-5. See FIG. 3. The solubility of the protein at intermediate pH 5.5-6.0 is ˜60-70%. Therefore the solubility profile of the legumin-type globulin accumulated in vpe-quad seed is markedly altered compared to wild-type supporting a function of proteolytic processing in determining this physiochemical property.

III. Conclusions

A. Vegetative-Type VPE Expression in Developing Seed

A common theme of storage protein deposition in the PSV of plant seeds is pro-protein processing by proteolytic cleavage at Asn residues in the P1 position of cleavage sites. Prior to the present disclosure, vegetative-type VPE genes were not believed to be involved in Asn-specific storage protein processing because earlier studies strongly implied that vegetative-type VPE genes encode isoforms of VPE that are not expressed in seed, but are specific to vegetative tissues. The RT-PCR detection of significant amounts of γVPE message in developing seed of wild-type plants was therefore a surprising result. However, this result is firmly supported by the MPSS transcript profiles obtained for the VPE genes. Although the MPSS analysis corroborated prior reports of γVPE expression in leaf and βvpe expression in developing seed, it also clearly showed that expression of these VPE genes are not mutually exclusive to those tissues as previously implied. The present analysis identified expression of all four VPE genes in developing seed, with transcript levels of each VPE gene exceeding those measured in non-seed tissues (root, leaf, shoot inflorescences).

B. Functions of VPE Genes

Interestingly, the expression patterns of the VPE genes appear to be significantly different from each other, yet at least three of the four genes in Arabidopsis seem to be involved in seed storage protein processing. It may expected that VPE gene functions are difficult to identify in many cases from single or even double mutants as overlapping or induced expression will act in a compensatory fashion similar to what we observed with single gene VPE mutants in seed protein processing. However, this would not be expected to occur in the vpe-quad mutant for which all VPE genes identified in the Arabidopsis genome are knocked out, and in fact is confirmed by examination of seed protein processing in this report. Surprisingly, despite VPE being implicated in several processes throughout plant growth and development, no deleterious or pleiotropic effects of not having a functional VPE protease were detected.

C. Seed Proteins are Processed by Vegetative-Type VPE

To measure the specific contribution of αVPE and γVPE to storage protein processing it was necessary to obtain seed from plants homozygous for additional combinations of VPE mutant alleles. Investigation of the seed protein profiles from either βvpe/γVPE or αype/βvpe/γVPE clearly identified increased accumulation of legumin-type globulin precursors indicating that both seed- and vegetative-type VPE can perform roles in storage protein processing. Additionally, no wild-type α- or β-chains of legumin-type globulins could be identified in seed devoid of αype, βvpe and γVPE supporting the hypothesis that VPEs are unique in their responsibility to process legumin-type globulin storage proteins at the conserved Asn-Gly peptide bond separating the chains. Furthermore, this exclusive responsibility extends to Asn-specific napin-type albumin processing as no wild-type small chains were found in vpe-quad. Also, similar to what was reported for βvpe, no evidence linking a specific VPE gene to proteolytic processing of a specific subset of legumin-type or napin-type storage proteins was found. Therefore, both the in planta functional analysis of VPE mutant Arabidopsis plants and the VPE gene expression analysis does not support the paradigm of two strict VPE classes, seed-type and vegetative-type, performing entirely separate functions as previously proposed. Instead, evidence presented here suggests that VPE gene family members have multiple expression patterns, and overlapping functions in at least developing seed.

D. Processing and Storage Protein Accumulation Mechanisms

Mature VPE-processed legumin-type globulin from soybean (glycinin) is considerable less soluble under acidic conditions at pH 4-6 when compared to bacterially expressed precursors of glycinin. VPE-processed Arabidopsis legumin-type globulins are also mostly insoluble at pH 5.5-6, which coincides with the pH of the PSV in developing seed. Although, alternatively processed legumin-type globulins in vpe-quad appear to be partial soluble at pH 5.5, they are insoluble under more acidic conditions. These data show that the specific solubility properties are impacted by the processing status of legumin-type globulin polypeptides. Recently it has been shown that an intermediate form of a drought responsive cysteine protease (iRD21) is insoluble under acidic conditions and is forming aggregates in vacuoles. Further, it has been suggested, that this aggregate may functions as a stock of inactive protease that could be made soluble under the appropriate physiological conditions to be available as an active enzyme. Similar to iRD21, aggregation of globulins in PSV, perhaps induced by limited proteolytic processing, could serve as a mechanism to ensure long-term stable globulin storage by sequestering these proteins away from the lytic conditions of the vacuole. During germination, storage proteins could be mobilized from these aggregates by a change of the pH or of the ionic strength of the vacuole, which would render the proteins soluble and make them accessible to proteolytic enzymes.

Inhibition of the Expression of Vacuolar Processing Enzymes in Soybean

A. Soybean plants with reduced vacuolar processing enzyme expression in seed were produced by transformation of plants with expression cassettes designed to knock down expression of the endogenous VPE genes in seed. Two different expression cassettes were each designed and used to independently accomplish this task, one cassette utilized an hpRNA construct in which DNA fragments corresponding to the sequence of the endogenous VPE genes being suppressed is cloned in a loop between two complementary DNA sequences (EL hpRNA; see WO 0200904). The second cassette consisted of an intron-spliced self-complimentary hairpin RNAi (ihpRNA) construct (Smith et al. (2000) Nature 407: 319-320) designed such that final cassette consisted of two identical ihpRNAs each expressed using an independent promoter.

The loop sequence of the EL hpRNA expression cassette was constructed using standard cloning techniques to splice rtPCR-amplified fragments (293-570 base pairs) of each of the soy VPE genes (Vpe1a, Vpe1b, Vpe2a, Vpe2b, Vpe3a) together in the same sense orientation. The EL hpRNA cassette was then constructed by linking the Kuntz trypsin inhibitor (KTI) promoter (nucleotides 5-2086 of NCBI Accession No. AF233296) the EL DNA sequence, the loop sequence of VPE genes in sense orientation, the EL DNA sequence in reverse orientation (complementary), and the KTI transcriptional termination sequence (nucleotides 2740-2927 of NCBI Accession No. AF233296). SEQ ID NO:15 shows the sequence of this expression cassette.

The stem sequence of the ihpRNA expression cassette was constructed using standard cloning techniques to splice rtPCR-amplified fragments of each of the soy VPE genes (Vpe1a, Vpe1b, Vpe2a, Vpe2b, Vpe3a, and Vpe3b) together. One transcriptional unit of the ihpRNA cassette was then constructed by linking the KTI promoter with the stem sequence fragment in the sense orientation, a PCR-amplified FAD2 intron sequence (nucleotides 142-1274 of NCBI Accession No. AC069473), and the same stem sequence fragment in reverse orientation. The second transcriptional unit of the ihpRNA cassette was constructed in the same fashion with the exception that the late seed preferred (LSP) promoter is substituted for the KTI promoter. The completed ihpRNA expression cassette contained both of these transcriptional units.

Soybean embryos are transformed with the expression cassettes described. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos that produce secondary embryos are then excised and placed into a suitable liquid medium. After repeated selection for clusters of somatic embryos that multiplied as early, globular staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327: 70-73, U.S. Pat. No. 4,945,050). A DuPont Biolistic PDS1000/HE instrument (helium retrofit) can be used for these transformations.

A selectable marker gene which can be used to facilitate soybean transformation is a transgene composed of the ³⁵S promoter from Cauliflower Mosaic Virus (Odell et al. (1985) Nature 313: 810-812), the hygromycin phosphotransferase gene from plasmid pJR225 (from E. coli; Gritz et al. (1983) Gene 25: 179-188) and the 3′ region of the nopaline synthase gene from the T-DNA of the Ti plasmid of Agrobacterium tumefaciens. The seed expression cassette comprising the phaseolin 5′ region, the fragment encoding the RNA suppression molecule and or the polypeptide of interest and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds, and the supernatant removed. The DNA-coated particles are then washed once in 400 μL 70% ethanol and resuspended in 40 μL of anhydrous ethanol. The DNA/particle suspension can be sonicated three times for one second each. Five μl of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

B. Soybean plants that were genetically modified to reduce the activity of vacuolar processing enzymes were produced by transforming soybean with a gene silencing vector, KS217, designed to reduce the activity of five soybean vacuolar processing enzymes. The KS217 vector had a VPE cassette containing sequences corresponding to fragments of the miRNA sequences of the five soybean VPE's shown below:

| Soybean | |
|---|---|
| VPE | Nucleotide Sequence Used for KS217 Vector |
| VPE1 | nucleotides 1-292 of SEQ ID NO: 11 |
| VPE1b | nucleotides 12-137 and 1428-1678 of SEQ ID NO: 13 |
| VPE2 | nucleotides 1-544 of SEQ ID NO: 5 |
| VPE2b | nucleotides 1181-1694 of SEQ ID NO: 7 |
| VPE3 | nucleotides 1273-1565 of SEQ ID NO: 9 |

The KS217 vector was constructed with a sense sequence upstream of the VPE cassette, and an inverted repeat of this sense sequence downstream of the VPE cassette.

Soybean embryonic suspension cultures were transformed with the KS217 vector by particle bombardment essentially as described above. The embryos were selected based on the expression of a selectable marker gene, and then regenerated into fertile transgenic soybean plants. Protein was extracted from seeds from these plants, and analyzed by SDS-PAGE. More than 50% of the soybean storage protein glycinin in the transformed seeds accumulated as proglycinin precursor, and this phenotype was found to be stable over at least three generations. The alteration in glycinin processing demonstrates that transformation with the KS217 vector successfully reduced the expression of the corresponding soybean VPE's.

Inhibition of the Expression of Vacuolar Processing Enzymes in Arabidopsis

Plants that were genetically modified to reduce the activity of α-vacuolar processing enzyme, β-vacuolar processing enzyme, γ-vacuolar processing enzyme, ε-vacuolar processing enzyme and three aspartic proteases. These plants were produced by transforming an Arabidopsis line containing knock-out mutations in α-VPE, β-VPE, γ-VPE, and ε-VPE (the “vpe-quad mutant”; see Gruis et al. (2004) Plant Cell 16: 270-90) with a gene silencing vector designed to reduce the activity of three different Arabidopsis aspartic proteases (the “AP1-2-3 RNAi vector”). The AP1-2-3 RNAi vector contained sequences corresponding to the following fragments of the Arabidopsis aspartic protease mRNA sequences:

| NCBI | Fragment Used for |
|---|---|
| Accession Number | Gene Silencing Vector |
| NM_104909 | nucleotides 1377-1614 |
| NM_101062 | nucleotides 1341-1631 |
| NM_116684 | nucleotides 1234-1461 |

The AP-1-2-3 RNAi vector also contained an inverted repeat of this sense sequence, and an intron from the maize alcohol dehydrogenase gene (ADH1) in the spacer region between the sense sequence and the antisense sequence. The Arabidopsis vpe-quad mutant plants were transformed by the floral dip method with the AP 1-2-3 RNAi vector by Agrobacterium-mediated transformation as described by Clough and Bent (1998) Plant J. 16: 735-43. After self-pollination, hemizygous transgenic seedlings underwent selection based on the expression of a selectable marker gene. The integration of the AP 1-2-3 RNAi cassette into the plant genome was confirmed by PCR with primer pairs that amplified a fragment of the RNAi cassette and a fragment of the selectable marker gene. Transgenic plants were then allowed to self-pollinate and the genetic transmission of the transgene was confirmed by selection of transgenic seedlings based on the selectable marker gene.

Protein was extracted from segregating single hemizygous and homozygous transgenic and wild type seeds, and analyzed by SDS-PAGE. Approximately 50-75% of the seeds collected from several independent transgenic events showed reduced processing of the seed albumin (diminished presence of large and small albumin chains and accumulation of albumin pro-protein precursor) consistent with the expected semi-dominant/dominant action of the AP silencing cassette. Suppression of albumin processing was not observed in single seed transgenic events in control vpe-quad plants that were transformed with a vector lacking the AP1-2-3 RNAi cassette. The alteration in seed protein processing in the plants transformed with the AP-1-2-3 RNAi cassette demonstrates that this cassette reduced the expression of the corresponding Arabidopsis proteases.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A soybean plant that is genetically modified to alter one or more functional properties of one or more seed storage proteins, wherein said soybean plant is genetically modified to reduce or eliminate the activity of one or more vacuolar processing enzymes in its seed.

2. The plant of claim 1, wherein said soybean plant is stably transformed with at least one expression cassette capable of expressing a polynucleotide that inhibits the expression of a vacuolar processing enzyme in seed.

3. The soybean plant of claim 1, wherein said soybean plant is genetically modified to reduce or eliminate the proteolytic activity of two or more vacuolar processing enzymes in its seed.

4. The plant of claim 3, wherein the plant is genetically modified to reduce or eliminate the proteolytic activity of three or more vacuolar processing enzymes in its seed.

5. The plant of claim 4, wherein the plant is genetically modified to inhibit the expression of four or more vacuolar processing enzymes in its seed.

6. The plant of claim 1, wherein at least one vacuolar processing enzyme is selected from the group consisting of Vpe1a, Vpe1b, Vpe2a, Vpe2b, Vpe3a, and Vpe3b.

7. The plant of claim 8, wherein at least one vacuolar processing enzyme is selected from the group consisting of SEQ ID NO:2, SEQ ID NO:4, SEQ ID NO:6, SEQ ID NO:8, SEQ ID NO:10, SEQ ID NO:12, and SEQ ID NO:14.

8. The plant of claim 1, wherein said soybean plant is stably transformed with at least one expression cassette comprising a polynucleotide encoding a polypeptide that inhibits the proteolytic activity of a vacuolar processing enzyme in seed.

9. The plant of claim 8, wherein said polypeptide that inhibits the proteolytic activity of a vacuolar processing enzyme is an antibody that binds to one or more soybean vacuolar processing enzymes.

10. The plant of claim 8, wherein said polypeptide that inhibits the proteolytic activity of a vacuolar processing enzyme is a polypeptide that specifically inhibits the activity of one or more vacuolar processing enzymes.

11. The plant of claim 1, wherein at least one of said seed storage proteins is selected from the group consisting of globulins and albumins.

12. The plant of claim 11, wherein at least one of said seed storage proteins is glycinin.

13. Transgenic seed of the plant of claim 1.

14. A method for producing a soybean seed storage protein having one or more altered functional properties, said method comprising the steps of
(a) transforming a soybean plant cell with at least one expression cassette capable of expressing a polynucleotide that reduces or eliminates the activity of at least one vacuolar processing enzyme in the seed of said soybean plant;
(b) regenerating a transformed plant from the transformed plant cell of step (a); and
(c) collecting seed from the transformed plant of step (b).

15. The method of claim 14, wherein the activity of at least two vacuolar processing enzymes is reduced or eliminated in the seed of said plant.

16. The method of claim 15, wherein the activity of at least two vacuolar processing enzymes is reduced or eliminated in the seed of said plant.

17. The method of claim 16, wherein the activity of at least two vacuolar processing enzymes is reduced or eliminated in the seed of said plant.

18. The method of claim 14, wherein at least one vacuolar processing enzyme is selected from the group consisting of Vpe1a, Vpe1b, Vpe2a, Vpe2b, Vpe3a, and Vpe3b.

19. The method of claim 18, wherein at least one vacuolar processing enzyme is selected from the group consisting of SEQ ID NO:2, SEQ ID NO:4, SEQ ID NO:6, SEQ ID NO:8, SEQ ID NO:10, SEQ ID NO:12, and SEQ ID NO:14.

20. The method of claim 14, wherein at least one altered functional property is solubility of the seed storage protein.

21. The method of claim 20, wherein the solubility of at least one seed storage protein is increased at low pH.

22. The method of claim 21, wherein the solubility of the seed storage protein is increased between pH 4.0 and 6.0.

23. The method of claim 14, wherein at least one seed storage protein is selected from the group consisting of glycinin and 2S-albumin.

24. The method of claim 23, wherein said seed storage protein is glycinin.

25. The method of claim 14, wherein the expression cassette capable of expressing a polynucleotide that reduces or eliminates the activity of a vacuolar processing enzyme in the seed of said soybean plant comprises:
(a) a sense sequence consisting of at least 19 nucleotides corresponding to an mRNA encoding a soybean vacuolar processing enzyme; and
(b) a complementary nucleotide sequence having at least 94% identity to the complement of the sense sequence of (a).

26. The method of claim 25, wherein the expression cassette capable of expressing a polynucleotide that reduces or eliminates the activity of a vacuolar processing enzyme in the seed of said soybean plant comprises a loop sequence operably linked to the sense sequence and the complementary nucleotide sequence.

27. The method of claim 26, wherein said loop sequence additionally comprises an intron that is capable of being spliced in a soybean seed.

28. The method of claim 25, wherein said soybean vacuolar processing enzyme is selected from the group consisting of Vpe1a, Vpe1b, Vpe2a, Vpe2b, Vpe3a, and Vpe3b.

29. The method of claim 28, wherein said sense sequence consists of at least 19 nucleotides of a nucleotide sequence selected from the group consisting of SEQ ID NO:1, SEQ ID NO:3, SEQ ID NO:5, SEQ ID NO:7, SEQ ID NO:9, SEQ ID NO:11, and SEQ ID NO:13.

30. The method of claim 14, wherein the expression cassette capable of expressing a polynucleotide that reduces or eliminates the activity of a vacuolar processing enzyme in the seed of said soybean plant comprises a sense sequence consisting of at least 19 nucleotides corresponding to a messenger RNA encoding a soybean vacuolar processing enzyme.

31. The method of claim 30, wherein said soybean vacuolar processing enzyme is selected from the group consisting of Vpe1a, Vpe1b, Vpe2a, Vpe2b, Vpe3a, and Vpe3b.

32. The method of claim 31, wherein said sense sequence consists of at least 19 nucleotides of a nucleotide sequence selected from the group consisting of SEQ ID NO:1, SEQ ID NO:3, SEQ ID NO:5, SEQ ID NO:7, SEQ ID NO:9, SEQ ID NO:11, and SEQ ID NO:13.

33. The method of claim 30, wherein said soybean plant is stably transformed to express an complementary nucleotide sequence having at least 94% identity to the complement of the sense sequence.

34. The method of claim 33, wherein said sense sequence and said complementary nucleotide sequence are comprised within the same expression cassette.

35. The method of claim 33, wherein said sense sequence and said complementary nucleotide sequence are comprised within different expression cassettes.

36. The method of claim 14, wherein the expression cassette capable of expressing a polynucleotide that reduces or eliminates the activity of a vacuolar processing enzyme in the seed of said soybean plant comprises a complementary nucleotide sequence having at least 94% identity to the complement of a sense sequence consisting of at least 19 nucleotides of a DNA sequence corresponding to a messenger RNA for a soybean vacuolar processing enzyme.

37. The method of claim 14, wherein the expression cassette capable of expressing a polynucleotide that reduces or eliminates the activity of a vacuolar processing enzyme in the seed of said soybean plant comprises:
(a) a sense sequence consisting of at least 50 nucleotides of a sequence that is not endogenously expressed in soybean.
(b) a complementary nucleotide sequence having at least 94% identity to the complement of the sense sequence of (a); and
(c) a loop sequence positioned on the 3′ end of the sense sequence and the 5′end of the complementary nucleotide sequence, wherein the loop sequence comprises at least 50 contiguous nucleotides corresponding to a messenger RNA encoding a soybean vacuolar processing enzyme.

38. A transformed soybean plant produced according to the method of claim 14.

39. A composition comprising at least one soybean seed storage protein produced according to the method of claim 14.

40. A method for producing a soybean seed storage protein having one or more altered functional properties, said method comprising the steps of
(a) transforming a soybean plant cell with at least one expression cassette comprising a polynucleotide encoding a polypeptide that reduces or eliminates the activity of at least one vacuolar processing enzyme in seed.
(b) regenerating a transformed plant from the transformed plant cell of step (a); and
(c) collecting seed from the transformed plant of step (b).

41. The method of claim 40, wherein said polypeptide that inhibits the enzymatic activity of a vacuolar processing enzyme is an antibody that binds to one or more soybean vacuolar processing enzymes.

42. The method of claim 40, wherein said polypeptide that inhibits the enzymatic activity of a vacuolar processing enzyme is a polypeptide that inhibits the proteolytic activity of one or more soybean vacuolar processing enzymes.

43. A transformed soybean plant produced according to the method of claim 39.

44. A composition comprising at least one soybean seed storage protein produced according to the method of claim 39.

45. An isolated polypeptide comprising an amino acid sequence selected from the group consisting of:
(a) the amino acid sequence comprising SEQ ID NO: 2, 4, 6, 8, or 10;
(b) an amino acid sequence comprising at least 90% sequence identity to SEQ ID NO: 6, 8, or 10 wherein said polypeptide has protease activity; and
(c) an amino acid sequence comprising at least 100 consecutive amino acids of SEQ ID NO:6, 8, or 10, wherein said polypeptide retains protease activity.

46. An isolated polynucleotide comprising a nucleotide sequence encoding a polypeptide of claim 45.

47. An expression cassette comprising the polynucleotide of claim 46.

48. The expression cassette of claim 47, wherein said polynucleotide is operably linked to a promoter that drives expression in a plant.


SEQUENCE LISTING (15 sequences)

SEQ ID NO: 1 - DNA, 1769 bp, Glycine max
gcacgagccctgttcctgtgtgtgtgagtgaccgagtgagtttgtttttctcagctgatatatatggcgcttgatcgctccattataagcaaaacgacgtggtacagcgtcgtattatggatgatggtggtgctggtgagagtgcacggtgcagccgcgaggccgaaccggaaggagtgggactcagtcataaagttaccgactgaaccggtggatgctgactcggatgaagtgggaacacgatgggcggttctcgtggctggttcaaacggctacggaaactacaggcatcaagcagatgtgtgccatgcgtaccagttgctgataaaaggtggactaaaagaagagaacatagtggtgtttatgtacgatgacatagctaccaacgagttgaatcctagacatggagtcatcatcaaccaccctgagggagaagatctgtatgctggtgttcctaaggattacaccggtgataatgtgacgacggagaacctctttgctgttattcttggagacaagagtaaattgaagggaggaagtggcaaagtgatcaacagcaaacccgaggacagaatatttatatactactctgatcatggaggtcctggaatacttgggatgccaaacatgccatacctttatgccatggattttattgatgtcttgaagaagaaacatgcatctggaagttacaaggagatggttatatacgtggaagcttgtgaaagtgggagcgtgtttgagggtataatgcctaaggatctgaatatttatgtcacaactgcatcaaatgcacaagagaatagttggggaacttattgtcctggaatggatccttctccacctccagagtacatcacttgcctaggggatttgtacagcgttgcttggatggaagatagtgaggctcacaatctaaaaagggaatccgtgaaacaacaatacaaatcggtaaagcaacggacttcaaatttcaacaactatgcgatgggttctcatgtgatgcaatatggtgataccaacatcacagctgaaaagctttatttataccaaggttttgatcctgccactgtgaacttccctccacaaaacggcaggctagaaactaaaatggaagttgttaaccaaagagatgcagaacttttgttcatgtggcaaatgtatcagagatcaaaccatcagtcagaaaataagacagacatcctcaaacaaattgcggagacagtgaagcataggaaacacatagatggtagcgtggaattgattggagttttactgtatggaccaggaaaaggttcttctgttctacaatccgtgagggctcctggttcgtcccttgttgatgactggacatgcctaaaatcaatggttcgggtgtttgaaactcactgtgggacactgactcagtatggcatgaaacacatgcgagcattcgccaacatttgcaacagtggcgtttctgaggcctccatggaagaggcttgtttggcagcctgtgaaggctacaatgctgggctattgcatccatcaaacagaggctacagtgcttgattttgggttttgtacacaaaagctttaaagcccggttgatgatgtaatatttctctattgcattctgcctactggtttctgctgcttgtgtcaaattttctctaaactagagtagcccaatagcatacgtgttatgtgcatgtgtcatgtatacaagtgtaatactaaaaccttctacataatataagattagttagtttaaaaaaaaaaaaaaaaaa

SEQ ID NO: 2 - protein, 495 aa, Glycine max
MALDRSIISKTTWYSVVLWMMVVLVRVHGAAARPNRKEWDSVIKLPTEPVDADSDEVGTRWAVLVAGSNGYGNYRHQADVCHAYQLLIKGGLKEENIVVFMYDDIATNELNPRHGVIINHPEGEDLYAGVPKDYTGDNVTTENLFAVILGDKSKLKGGSGKVINSKPEDRIFIYYSDHGGPGILGMPNMPYLYAMDFIDVLKKKHASGSYKEMVIYVEACESGSVFEGIMPKDLNIYVTTASNAQENSWGTYCPGMDPSPPPEYITCLGDLYSVAWMEDSEAHNLKRESVKQQYKSVKQRTSNFNNYAMGSHVMQYGDTNITAEKLYLYQGFDPATVNFPPQNGRLETKMEVVNQRDAELLFMWQMYQRSNHQSENKTDILKQIAETVKHRKHIDGSVELIGVLLYGPGKGSSVLQSVRAPGSSLVDDWTCLKSMVRVFETHCGTLTQYGMKHMRAFANICNSGVSEASMEEACLAACEGYNAGLLHPSNRGYSA

SEQ ID NO: 3 - DNA, 1806 bp, Glycine max
gcacgaggtgagtctttcttagctgatatggcggttgatcgctcccttacgaggtgctgtagcctcgtactgtggtcgtggatgttgctgaggatgatgatggcgcagggtgcagccgcgagggccaaccggaaggagtgggactcggtcataaagttaccggctgaaccggtcgatgctgactcggatcatgaagtgggaacacgatgggcggttcttgtggctggttcaaacggctatggaaactacaggcatcaagcagatgtgtgccatgcgtaccagttgctgataaaaggtgggctaaaagaagagaacatagtggtgtttatgtacgatgacatagctacagacgagttaaatcccagacctggagtcatcatcaaccaccctgagggacaagatgtgtatgctggtgttcctaaggattacaccggtgagaatgtgacggcccagaacctctttgccgttattcttggagacaagaataaagtgaagggaggaagtggcaaagtgatcaatagcaaacctgaggacagaatatttatatactactctgatcatggaggtccgggagttcttgggatgccaaacatgccatacctttatgctatggactttattgaagtcttgaagaagaaacatgcatctggaggttacaagaagatggtcatatacgtggaagcttgtgaaagtgggagcatgtttgagggtataatgcctaaggatctgcagatttatgtcacaactgcatccaatgcacaagagaatagttggggaacttattgtcctggaatggatccttctccacctccagagtacatcacttgcctaggggatttgtacagtgttgcttggatggaagatagtgagactcataatctaaaaagggagtccgtgaaacaacaatacaaatcggtaaagcaacggacttcaaatttcaacaactatgcgatgggttctcatgtgatgcaatacggtgacacaaacatcacagctgaaaagctttatttataccaaggttttgatcctgccgctgtgaacttccctccacagaacggaaggctagaaactaaaatggaagttgttaaccaaagagatgcagaacttttcttcatgtggcaaatgtatcagagatcaaaccatcagccagaaaagaagacagacatcctcaaacagatagcggagacagtgaagcataggaaacacatagatggtagcgtggaattgattggagttttattgtatggaccaggaaaaggttcttctgttctacaatccatgagggctcctggtcttgcccttgttgatgactggacatgcctaaaatcaatggttcgggtgtttgagactcactgtgggacactgactcagtatggcatgaaacacatgcgagcatttgccaacatttgcaacagcggtgtttctgaggcctccatggaagaggtttgtgtggcagcttgtgaaggctacgattctgggctattacatccatcaaacaaaggctatagtgcttgattttgggttttgtacacagcttaaaaacccggttgatgatgtaatacttctctattgcattctccctactggtttctgctgcatgtgtcaaattttctctaaactagagtagcccaatagcatacgtgttatgagcattggtcatgtatataagtgtaatagtaatatcttttacatattataagatcagttagtttggtttactagtgtctgtttcaagctctattttcttgaactcaactccttctaaatcaaggagatttttcttaaaaaaaaaaaaaaaaaaaaaaa

SEQ ID NO: 4 - protein, 495 aa, Glycine max
MAVDRSLTRCCSLVLWSWMLLRMMMAQGAAARANRKEWDSVIKLPAEPVDADSDHEVGTRWAVLVAGSNGYGNYRHQADVCHAYQLLIKGGLKEENIVVFMYDDIATDELNPRPGVIINHPEGQDVYAGVPKDYTGENVTAQNLFAVILGDKNKVKGGSGKVINSKPEDRIFIYYSDHGGPGVLGMPNMPYLYAMDFIEVLKKKHASGGYKKMVIYVEACESGSMFEGIMPKDLQIYVTTASNAQENSWGTYCPGMDPSPPPEYITCLGDLYSVAWMEDSETHNLKRESVKQQYKSVKQRTSNFNNYAMGSHVMQYGDTNITAEKLYLYQGFDPAAVNFPPQNGRLETKMEVVNQRDAELFFMWQMYQRSNHQPEKKTDILKQIAETVKHRKHIDGSVELIGVLLYGPGKGSSVLQSMRAPGLALVDDWTCLKSMVRVFETHCGTLTQYGMKHMRAFANICNSGVSEASMEEVCVAACEGYDSGLLHPSNKGYSA

SEQ ID NO: 5 - DNA, 1936 bp, Glycine max
gcacgagaattaaattaatagaggatgaaattctagtttaaggaaggttggttggttgggtgggggtaggagatactctcattcacctcccatcatcattataatcattcattccaacctacccttattcttcttcttcaatttcacacccatcatggaccgttttccgatcctctttctcgtcgccaccctcatcaccctcgcctccggtgcccgccacgatattctccggttaccctccgaagcttccaggttcttcaaagcacctgctaatgccgatcaaaacgatgagggcaccaggtgggccgttttagttgccggttccaatggctactggaattacaggcaccagtctgatgtttgccatgcatatcaactactgaggaaaggtggtgtgaaagaggaaaatattgttgtatttatgtatgatgacattgctttcaatgaagagaacccacggcctggagtcattattaacagtccacacggaaatgatgtttacaagggagttcctaaggattacgttggtgaagatgttactgttgacaacttttttgctgctatacttggaaataagtcagctcttactggtggcagtgggaaggttgtggatagtggccccaatgatcatatatttatatactactctgatcatggcggtccgggagtgctagggatgcctactaatccatacatgtatgcatccgatctgattgaagtcttgaagaagaagcatgcttctggaacttataaaagcctagtattttatctagaggcatgtgaatctgggagtatctttgaaggtcttcttccagaaggtctgaatatctatgcaacaacagcttcaaatgctgaagaaagcagttggggaacatattgtcctggggagtatcctagtcctccccctgaatatgaaacctgcctgggtgacctgtacagtgttgcttggatggaagatagtgacatacacaatttgcgaacagaaactttacatcaacaatacgacttggtcaaagaaaggactatgaatggaaattcaatctatggttcccacgtgatgcagtatggtgacatagggcttagcaagaacaatcttgtcttatatttgggtacaaatcctgctaatgataattttacttttgtgcataaaaactcattggtgccaccttcaaaagcagtcaaccaacgtgatgcagatctcatccatttctgggataagttccgcaaagctcctgtgggttcttctaggaaagctgcagctgagaaagaaattctggaagcaatgtctcacagaatgcatatagatgacaacatgaaacttattggaaagctcttatttggcattgaaaagggtccagaactgcttagcagtgttagacctgctgggcaaccacttgttgatgactgggactgccttaaaacactggttaggacttttgagacacattgtggatctctgtctcagtatgggatgaaacatatgaggtcctttgcaaacttctgcaacgctggaatacggaaagagcaaatggctgaggcctcggcacaagcatgtgtcagtatccctgcaagttcctggagttctctgcacaggggtttcagtgcataattcctagaatccgctccattgaagacagagtatagtcgttgtaacattattctttacgagcgttatgtactgtacctggacatgatttcttataccaaccctgttaataagcatgggacgctggggaaacctatttacattgtaatttcgtgcaaaatagatgctgtaacaaaggcattttacttttacttggggagaggcagtggaaccataaggaccttggaaattctgattaatatgacagggcacaatatcgtgtttgtaagccaacgctttatttttattttatggtaacccctttctgtggataaaaaaaaaaaaaaaaaa

SEQ ID NO: 6 - protein, 484 aa, Glycine max
MDRFPILFLVATLITLASGARHDILRLPSEASRFFKAPANADQNDEGTRWAVLVAGSNGYWNYRHQSDVCHAYQLLRKGGVKEENIVVFMYDDIAFNEENPRPGVIINSPHGNDVYKGVPKDYVGEDVTVDNFFAAILGNKSALTGGSGKVVDSGPNDHIFIYYSDHGGPGVLGMPTNPYMYASDLIEVLKKKHASGTYKSLVFYLEACESGSIFEGLLPEGLNIYATTASNAEESSWGTYCPGEYPSPPPEYETCLGDLYSVAWMEDSDIHNLRTETLHQQYDLVKERTMNGNSIYGSHVMQYGDIGLSKNNLVLYLGTNPANDNFTFVHKNSLVPPSKAVNQRDADLIHFWDKFRKAPVGSSRKAAAEKEILEAMSHRMHIDDNMKLIGKLLFGIEKGPELLSSVRPAGQPLVDDWDCLKTLVRTFETHCGSLSQYGMKHMRSFANFCNAGIRKEQMAEASAQACVSIPASSWSSLHRGFSA

SEQ ID NO: 7 - DNA, 1942 bp, Glycine max
gcacgagctctctctctctctctctctctctctctctctctctctctctctctctctctctctctctctctctctctctctctctctctctctctctctctctcctcactcgttcattccaacctacccttattcttcttcttcaattccacacccatcatggaccgttttccgatcctctttctcctcgccaccctcatcaccctcgcctccggtgcccgccacgatattctccggttaccctccgaagcatccacttttttcaaagcacccggtggcgatcaaaacgatgagggcacgaggtgggccgttttaattgccggttccaatggctactggaattacaggcaccagtctgatgtttgccatgcgtatcaactactgaggaaaggtggtctcaaagaagaaaatattgttgtatttatgtatgatgacattgctttcaacgaagagaacccgcgacctggagtcattattaacagtccacatggaaatgatgtttacaagggagtccctaaggattacattggtgaagatgtaactgttggcaacttttttgctgctatacttggaaataagtcagctcttactggtggcagtgggaaggttgtggatagtggtcccaatgatcatatatttatatattactctgatcatggcggtcctggagtgctagggatgcctactaatccatacatgtatgcatctgatctgattgaagtcttgaagaagaagcatgcttctggaagttataaaagcctagtattttatctagaggcatgtgaatctgggagtatctttgaaggtcttcttcctgaaggtctgaatatctatgcaacaacagcttcaaatgcagaagaaagcagttggggaacatattgtcctggggagtatcctagtcctccctctgaatatgaaacctgcctgggtgacctgtacagtgttgcttggatggaagacagtgacatacacaatttgcaaacagaaactttacatcaacaatacgaattggtcaaacaaaggactatgaatggaaattcaatttatggttcccacgtgatgcagtatggtgacatagggcttagcgagaacaatctcgtcttatatttgggtacaaatcctgctaatgataattttacttttgtgcttaaaaactcattggtgccaccttcaaaagcagtcaaccaacgtgatgcagatctcatccatttttgggataagttccgcaaagctcctgtgggttcttctaggaaagctgcagctgagaaacaaattcttgaagcaatgtctcacagaatgcatatagatgacagcatgaaacgtattggaaagctcttctttggcattgaaaagggtccagaactgcttagcagtgttagacctgctgggcaaccacttgttgatgactgggactgccttaaaacattggttaggacttttgagacacattgtggatccctgtctcagtatgggatgaaacatatgaggtcctttgcaaacttctgcaacgctggaatacgaaaagagcaaatggctgaggcctcagcacaagcatgtgtcaatatccctgctagttcctggagttctatgcacaggggtttcagtgcataattcctagaatgcgctccattgaagaccgagtatagtcgttgtaacattattctttacgagtgttatggactgtactctctgctcatgatttcttataccaaccctgtaaatacaaatgggacgctggggaaacctctttacattatagtttcctgcaaaatagatgctgtaacaaagacattttacttttacttggggagaggcagtggaaccataaggacccttggaacttctaattaatacgacagggcacaataccgtgtttgtaagccaacgctttgtttcaatttaatggtaaccccgttgtgtagaaaaaaaaaaaaaaaaaaaa

SEQ ID NO: 8 - protein, 483 aa, Glycine max
MDRFPILFLLATLITLASGARHDILRLPSEASTFFKAPGGDQNDEGTRWAVLIAGSNGYWNYRHQSDVCHAYQLLRKGGLKEENIVVFMYDDIAFNEENPRPGVIINSPHGNDVYKGVPKDYIGEDVTVGNFFAAILGNKSALTGGSGKVVDSGPNDHIFIYYSDHGGPGVLGMPTNPYMYASDLIEVLKKKHASGSYKSLVFYLEACESGSIFEGLLPEGLNIYATTASNAEESSWGTYCPGEYPSPPSEYETCLGDLYSVAWMEDSDIHNLQTETLHQQYELVKQRTMNGNSIYGSHVMQYGDIGLSENNLVLYLGTNPANDNFTFVLKNSLVPPSKAVNQRDADLIHFWDKFRKAPVGSSRKAAAEKQILEAMSHRMHIDDSMKRIGKLFFGIEKGPELLSSVRPAGQPLVDDWDCLKTLVRTFETHCGSLSQYGMKHMRSFANFCNAGIRKEQMAEASAQACVNIPASSWSSMHRGFSA

SEQ ID NO: 9 - DNA, 1948 bp, Glycine max
gcaccagaaaatgcccactttttttcttccaacgctcctcctccttctcatagccttcgccacctctgtctccggccgccgtgacctcgtcggagactttctccggctgccctccgaaactgataacgacgacaacttcaagggcacccggtgggccgtcctcctcgccggttccaatggttactggaattacagacatcaggctgatgtttgtcacgcctatcaaatattgaggaaaggtggtctgaaagaagaaaatattattgtttttatgtatgatgacattgcattcaatggggaaaacccaaggcctggagtcatcattaacaaaccagatggaggtgatgtttataaaggagttccaaaggattacaccggcgaagatgttactgttgataacttttttgctgctttacttggaaataagtcagcactgactggtggcagtgggaaggttgtggacagtggtcctgatgatcatatatttgtatactatactgaccatggaggtcctggggtgctcgggatgcctgctggtccttacttatacgcggatgatctgattgaagtcttgaagaaaaagcatgcttctggaacatataaaaacctagtattttatctggaggcatgtgaatctgggagtatctttgaaggtcttcttcctgaagatatcaatatttatgcaaccactgcttccaatgcagaagaaagtagttggggaacatattgccccggggagtatcctagtcctcccccagaatatacaacctgtttgggtgacttgtacagtgttgcttggatggaagacagtgacagacacaatttgcgaacagaaactctgcaccaacaatataaattggttaaagagaggactatatctggagattcatactatggctctcacgtgatgcagtatggtgatgtagggcttagcagagatgttctcttccattatttgggtacagatcctgctaatgataatttcacttttgtggatgaaaactccttatggtcaccttcaaaaccagtcaaccaacgtgatgctgatctcatccatttttgggataagttccgcaaagctcctgagggttctctcaggaaaaatacagctcagaaacaagttttggaagcaatgtctcacagaatgcatgtagacaacagtgtaaaactgattgggaagcttttatttggcattgaaaagggtccagaagtactcaacgctgttagaccggctggatcggcacttgttgatgactggcactgcctgaaaaccatggtgaggacttttgagacacattgtggatccttgtctcaatacgggatgaaacacatgaggtcctttgcaaacatctgcaatgtagggataaagaatgaacaaatggctgaggcttcagcacaagcttgtgtcagtattccttccaatccctggagttctctgcaaaggggtttcagtgcataataactccctgtaatgtgcactagtaaagaccaaagtatgattattgttacattatgttacatggttgtacttgtatatacatatcttgtcccacctttgtaaatacaattgggacactactaggattgggaagaagggtctttacatttatagtttggcaaatagatattgcaactacctttgtataattctatttctgaagaagcaattacaatttacaagggatggtgccatttacggcataaggattaaggagggataaagggaccaattgctttggaatatccactcattacaatgcatgtatgacaacacatagtaatatgatgtgtgtttttattcagtgggcaactggcagatcgggttttccctggtcacttttgtataattattccggaagaatttatgatgccaaaattattgtttaatattaatgacaacttgtatttatttttgtaaaaaaaaaaaaaaaaaaaaaaaaaaaaaaaa

SEQ ID NO: 10 - protein, 482 aa, Glycine max
MPTFFLPTLLLLLIAFATSVSGRRDLVGDFLRLPSETDNDDNFKGTRWAVLLAGSNGYWNYRHQADVCHAYQILRKGGLKEENIIVFMYDDIAFNGENPRPGVIINKPDGGDVYKGVPKDYTGEDVTVDNFFAALLGNKSALTGGSGKVVDSGPDDHIFVYYTDHGGPGVLGMPAGPYLYADDLIEVLKKKHASGTYKNLVFYLEACESGSIFEGLLPEDINIYATTASNAEESSWGTYCPGEYPSPPPEYTTCLGDLYSVAWMEDSDRHNLRTETLHQQYKLVKERTISGDSYYGSHVMQYGDVGLSRDVLFHYLGTDPANDNFTFVDENSLWSPSKPVNQRDADLIHFWDKFRKAPEGSLRKNTAQKQVLEAMSHRMHVDNSVKLIGKLLFGIEKGPEVLNAVRPAGSALVDDWHCLKTMVRTFETHCGSLSQYGMKHMRSFANICNVGIKNEQMAEASAQACVSIPSNPWSSLQRGFSA

SEQ ID NO: 11 - DNA, 1736 bp, Glycine max
  CDS (41)...(1528)
gtgagtgaccgagtgagtttgtttttctcagctgatatatatggcgcttgatcgctccattataagcaaaacgacgtggtacagcgtcgtattatggatgatggtggtgctggtgagagtgcacggtgcagccgcgaggccgaaccggaaggagtgggactcagtcataaagttaccgactgaaccggtggatgctgactcggatgaagtgggaacacgatgggcggttctcgtggctggttcaaacggctacggaaactacaggcatcaagcagatgtgtgccatgcgtaccagttgctgataaaaggtggactaaaagaagagaacatagtggtgtttatgtacgatgacatagctaccaacgagttgaatcctagacatggagtcatcatcaaccaccctgagggagaagatctgtatgctggtgttcctaaggattacaccggtgataatgtgacgacggagaacctctttgctgttattcttggagacaagagtaaattgaagggaggaagtggcaaagtgatcaacagcaaacccgaggacagaatatttatatactactctgatcatggaggtcctggaatacttgggatgccaaacatgccatacctttatgccatggattttattgatgtcttgaagaagaaacatgcatctggaagttacaaggagatggttatatacgtggaagcttgtgaaagtgggagcgtgtttgagggtataatgcctaaggatctgaatatttatgtcacaactgcatcaaatgcacaagagaatagttgggggacttattgtcctggaatggatccttctccacctccagagtacatcacttgcctaggggatttgtacagcgttgcttggatggaagatagtgaggctcacaatctaaaaagggaatccgtgaaacaacaatacaaatcggtaaagcaacggacttcaaatttcaacaactatgcgatgggttctcatgtgatgcaatatggtgataccaacatcacagctgaaaagctttatttataccaaggttttgatcctgccactgtgaacttccctccacaaaacggcaggctagaaactaaaatggaagttgttaaccaaagagatgcagaacttttcttattgtggcaaatgtatcagagatcaaaccatcagtcagaaaataagacagacatcctcaaacaaattgcggagacagtgaagcataggaaacacatagatggtagcgtggaattgattggagttttactgtatggaccaggaaaaggttcttctgttctacaatccgtgagggctcctggttcgtcccttgttgatgactggacatgcctaaaatcaatggttcgggtgtttgaaactcactgtgggacactgactcagtatggcatgaaacacatgcgagcattcgccaacatttgcaacagtggcgtttctgaggcctccatggaagaggcttgtttggcagcctgtgaaggctacaatgctgggctattccatccatcaaacagaggctacagtgcttgattttgggttttgtacacaaaagctttaaagcccggttgatgatgtaatatttctctattgcattctgcctactggtttctgctgcttgtgtcaaattttctctaaactagagtagcccaatagcatacgtgttatgtgcattggtcatgtatacaagtgtaatactaataccttcctacataatataagattagttagtttacttgtc

SEQ ID NO: 12 - protein, 495 aa, Glycine max
MALDRSIISKTTWYSVVLWMMVVLVRVHGAAARPNRKEWDSVIKLPTEPVDADSDEVGTRWAVLVAGSNGYGNYRHQADVCHAYQLLIKGGLKEENIVVFMYDDIATNELNPRHGVIINHPEGEDLYAGVPKDYTGDNVTTENLFAVILGDKSKLKGGSGKVINSKPEDRIFIYYSDHGGPGILGMPNMPYLYAMDFIDVLKKKHASGSYKEMVIYVEACESGSVFEGIMPKDLNIYVTTASNAQENSWGTYCPGMDPSPPPEYITCLGDLYSVAWMEDSEAHNLKRESVKQQYKSVKQRTSNFNNYAMGSHVMQYGDTNITAEKLYLYQGFDPATVNFPPQNGRLETKMEVVNQRDAELFLLWQMYQRSNHQSENKTDILKQIAETVKHRKHIDGSVELIGVLLYGPGKGSSVLQSVRAPGSSLVDDWTCLKSMVRVFETHCGTLTQYGMKHMRAFANICNSGVSEASMEEACLAACEGYNAGLFHPSNRGYSA

SEQ ID NO: 13 - DNA, 1715 bp, Glycine max
  CDS (19)...(1509)
tgttgctgtcgagctgatatggcggttgatcgctcccttacgaggtgctgtagcctcgtactgtggtcgtggatgttgctgaggatgatgatggcgcagggtgcagccgcgagggccaaccggaaggagtgggactcggtcataaagttaccggctgaaccggtcgatgctgactcggatcatgaagtgggaacacgatgggcggttcttgtggctggttcaaacggctatggaaactacaggcatcaagcagatgtgtgccatgcgtaccagttgctgataaaaggtgggctaaaagaagagaacatagtggtgtttatgtacgatgacatagctacagacgagttaaatcccagacctggagtcatcatcaaccaccctgagggacaagatgtgtatgctggtgttcctaaggattacaccggtgagaatgtgacggcccagaacctctttgccgttattcttggagacaagaataaagtgaagggaggaagtggcaaagtgatcaatagcaaacctgaggacagaatatttatatactactctgatcatggaggtccgggagttcttgggatgccaaacatgccatacctttatgctatggactttattgaagtcttgaagaagaaacatgcatctggaggttacaagaagatggtcatatacgtggaagcttgtgaaagtgggaaccatgttttgaagggtataatgcctaaggatctgcagatttatgtcacaactgcatcaaatgcacaagagaatagttggggaacttattgtcctggaatggatccttctccacctccagagtacatcacttgcctaggggatttgtacagtgttgcttggatggaagatagtgagactcataatctaaaaagggagtccgtgaaacaacaatacaaatcggtaaagcaacggacttcaaatttcaacaactatgcgatgggttctcatgtgatgcaatacggtgacacaaacatcacagctgaaaagctttatttataccaaggttttgatcctgccgctgtgaacttccctccacagaacggaaggctagaaactaaaatggaagttgttaaccaaagagatgcagaacttttcttcatgtggcaaatgtatcagagatcaaaccatcagccagaaaagaagacagacatcctcaaacagatagcggagacagtgaagcataggaaacacatagatggtagcgtggaattgattggagttttattgtatggaccaggaaaaggttcttctgttctacaatccatgagggctcctggtcttgcccttgttgatgactggacatgcctaaaatcaatggttcgggtgtttgagactcactgtgggacactgactcagtatggcatgaaacacatgcgagcatttgccaacatttgcaacagcggtgtttctgaggcctccatggaagaggtttgtgtggcagcttgtgaaggctacgattctgggctattacatccatcaaacaaaggctatagtgcttgattttgggttttgtacacagcttaaaaacccggttgatgatgtaatacttctctattgcattctccctactggtttctgctgcatgtgtcaaattttctctaaactagagtagcccaatagcatacgtgttatgagcattggtcatgtatataagtgtaatagtaatatcttttacatattataagatcagttagtttggtttacta

SEQ ID NO: 14 - protein, 496 aa, Glycine max
MAVDRSLTRCCSLVLWSWMLLRMMMAQGAAARANRKEWDSVIKLPAEPVDADSDHEVGTRWAVLVAGSNGYGNYRHQADVCHAYQLLIKGGLKEENIVVFMYDDIATDELNPRPGVIINHPEGQDVYAGVPKDYTGENVTAQNLFAVILGDKNKVKGGSGKVINSKPEDRIFIYYSDHGGPGVLGMPNMPYLYAMDFIEVLKKKHASGGYKKMVIYVEACESGNHVLKGIMPKDLQIYVTTASNAQENSWGTYCPGMDPSPPPEYITCLGDLYSVAWMEDSETHNLKRESVKQQYKSVKQRTSNFNNYAMGSHVMQYGDTNITAEKLYLYQGFDPAAVNFPPQNGRLETKMEVVNQRDAELFFMWQMYQRSNHQPEKKTDILKQIAETVKHRKHIDGSVELIGVLLYGPGKGSSVLQSMRAPGLALVDDWTCLKSMVRVFETHCGTLTQYGMKHMRAFANICNSGVSEASMEEVCVAACEGYDSGLLHPSNKGYSA

SEQ ID NO: 15 - DNA, 7825 bp, Artificial Sequence
  Expression cassette for suppression of soybean VPE expression
gggcgaattgggttacccggaccggaattcgagctcggtacccggggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccgctctagaactagtggatcccccgggctgcaggaattcgatgcacgagaattaaattaatagaggatgaaattctagtttaaggaaggttggttggttgggtgggggtaggagatactctcattcacctcccatcatcattataatcattcattccaacctacccttattcttcttcttcaatttcacacccatcatggaccgttttccgatcctctttctcgtcgccaccctcatcaccctcgcctccggtgcccgccacgatattctccggttaccctccgaagcttccaggttcttcaaagcacctgctaatgccgatcaaaacgatgagggcaccaggtgggccgttttagttgccggttccaatggctactggaattacaggcaccagtctgatgtttgccatgcatatcaactactgaggaaaggtggtgtgaaagaggaaaatattgttgtatttatgtatgatgacattgctttcaatgaagagaacccacggcctggagtcattattaacagtccacacggaaatgatgtttacaagggagttcctaaggattacgttggtgaagatgttactgttaaccaacgtgatgcagatctcatccatttttgggataagttccgcaaagctcctgtgggttcttctaggaaagctgcagctgagaaacaaattcttgaagcaatgtctcacagaatgcatatagatgacagcatgaaacgtattggaaagctcttctttggcattgaaaagggtccagaactgcttagcagtgttagacctgctgggcaaccacttgttgatgactgggactgccttaaaacattggttaggacttttgagacacattgtggatccctgtctcagtatgggatgaaacatatgaggtcctttgcaaacttctgcaacgctggaatacgaaaagagcaaatggctgaggcctcagcacaagcatgtgtcaatatccctgctagttcctggagttctatgcacaggggtttcagtgcataattcctagaatgcgctccattgaagaccgagtatagtcgttgtaacattattctttacgagtgttatggactgtactctctgctcatggtgaggacttttgagacacattgtggatccttgtctcaatacgggatgaaacacatgaggtcctttgcaaacatctgcaatgtagggataaagaatgaacaaatggctgaggcttcagcacaagcttgtgtcagtattccttccaatccctggagttctctgcaaaggggtttcagtgcataataactccctgtaatgtgcactagtaaagaccaaagtatgattattgttacattatgttacatggttgtacttgtatatacatatcttgtcccacctttgtaaatacaattcgatgggctgcaggaattcgatgtgagtgaccgagtgagtttgtttttctcagctgatatatatggcgcttgatcgctccattataagcaaaacgacgtggtacagcgtcgtattatggatgatggtggtgctggtgagagtgcacggtgcagccgcgaggccgaaccggaaggagtgggactcagtcataaagttaccgactgaaccggtggatgctgactcggatgaagtgggaacacgatgggcggttctcgtggctggttcaaacggctacggaaactacaggcatcaagcagatgtgtgccatgcgtaccagttgctgccacgaggtgagtctttcttagctgatatggcggttgatcgctcccttacgaggtgctgtagcctcgtactgtggtcgtggatgttgctgaggatgatgatggcgcagggtgcagccgcgagggccaaccggaaggagtgggactccatggaagaggtttgtgtggcagcttgtgaaggctacgattctgggctattacatccatcaaacaaaggctatagtgcttgattttgggttttgtacacagcttaaaaacccggttgatgatgtaatacttctctattgcattctccctactggtttctgctgcatgtgtcaaattttctctaaactagagtagcccaatagcatacgtgttatgagcattggtcatgtatataagtgtaatagtaatatcgactcgacgatgagcgagatgaccagctcgaattcatcactagtgaattcgcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacctcgagggggggccccggccgaagcttcggtccgggtcacccagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgcctataaatgggctcgcgataatgtcggccaatcaggtccgacaatctatcgattgtatgggaagcccgatgcgccagacttgtttctgaaacatggcaaaggtagccttgccaatgatgttacagatgagatggtcagactaaactgcctgacggaatttatgcctcttccgaccatcaagcattttatccgtactcctgatgatgcatggttactcaccactgcgatcccngggaaaacagcattccaggtattagaagaatatcctgattcaggtgaaaatattgttgatgcgctggcagtgttcctgcgccggttgcattcgattcctctttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataancttttgccattctcaccggattcagtcgtcactcatggtgatttctcacttgataaccttatttttgaccaggcgaaattaataggttgtattgatcttcgacgagtcggaatcgcagaccgataccaggatcttgccatcctatggaactgcctcggtgagttttctccttcattacagaaacggctttttcaaaaatatggtattgataatcctgatatgaataaattgcagtttcatttgatcctcgatgagtttttctaatcagaattggttaattggttgtaacactggcagagcattacgctgacttgacgggacggcggctttgttgaataaatcgaacttttgctgacttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgagccatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata